(19) United States
(12) Patent Application Publication
(10) Pub. No.: US 2025/0186699 A1
(43) Pub. Date: Jun. 12, 2025

(54) AUTOINJECTOR

(71) Applicant: Ypsomed AG, Burgdorf (CH)

(72) Inventors: Markus Tschirren, Burgdorf (CH); Christian Schrul, Oberburg (CH); Mario Bernhard, Burgdorf (CH); Nicolas Gruenig, Ostermundigen (CH); Marcel Allenspach, Burgdorf (CH); Gabriel Kalbermatter, Burgdorf (CH)

(21) Appl. No.: 19/055,232

(22) Filed: Feb. 17, 2025

Related U.S. Application Data
(63) Continuation of application No. PCT/EP2023/073281, filed on Aug. 24, 2023.

(30) Foreign Application Priority Data
Aug. 25, 2022 (EP) 22192171.1

(51) Int. Cl. A61M 5/20

(52) U.S. Cl. CPC A61M 5/2033; A61M 2205/8281

(57) ABSTRACT

An autoinjector including a drive device with a first housing part and a product container receiving device with a second housing part are connectable coaxially via a connection-side end portion on one of the two housing parts which is stepped in such a way that it can be inserted into the other of the two housing parts, and in the region of the end portion, a first snap-in connection and a second and third snap-in connection are provided, which can be snapped into place, one after the other, in the joining direction, and the first of the snap-in connections can be released against the joining direction, and at least the third or final snap-in connection is locked. Methods for the final assembly of the autoinjector involve establishing the snap-in connections for initially permitting release of the two housing parts and then ultimately locking them together.

### CROSS-REFERENCE TO RELATED APPLICATIONS

This application is a continuation of International Application No. PCT/EP2023/073281, filed on Aug. 24, 2023 entitled “IMPROVED AUTOINJECTOR” which in turn claims priority to European Patent Application No. 22192171.1, filed on Aug. 25, 2022 entitled “IMPROVED AUTOINJECTOR” each of which is incorporated by reference herein, in the entirety and for all purposes.

### TECHNICAL FIELD

The present disclosure relates to the field of medical injection devices for administering liquid medicaments, and more particularly relates to an autoinjector with an energy store for dispensing a predetermined dose from a product container that is used one time.

### BACKGROUND

Injection devices or injection apparatuses for the simplified administration of a substance inter alia comprise so-called autoinjectors which have an energy store with which the delivery process can be carried out automatically, i.e., without a force to be supplied or exerted externally by a user. The energy store advantageously stores the energy required for an automatic substance delivery in mechanical form. Such an energy store can be a spring which is installed in a tensioned state in the injection device and delivers energy by expansion. The energy is delivered to a piston rod or a pressure element, which pushes a piston into a product container. The energy store may also be provided in order to automate the process of inserting an injection needle. Alternatively, the insertion process can take place manually, i.e., exclusively by a user, without using energy stored in the injection device for this purpose.

The injection device may comprise a product container holder for receiving a product container, wherein the product container can be held in the product container holder radially, axially, and for instance also in a rotationally fixed manner. The product container holder may be connected to the housing of the injection device in an axially and rotationally fixed manner or may be movable relative to the housing during an insertion and/or needle retraction process. The product container may be a carpule for the repeatedly detachable connection to disposable injection needles or a disposable ready-to-use syringe with an injection needle non-detachably connected thereto. The product container has a hollow cylindrical product container portion which displaceably mounts a piston or plunger. The piston can form a sealing gap with the inner circumference of the product container portion and can be displaced in a distal direction by means of a piston rod in order to dispense product from the product container via the injection needle.

The injection device may have a needle protection sleeve which, after injection has taken place, projects distally beyond the distal end of the injection needle or is displaced relative to the housing into this position while expanding a needle protection sleeve spring in order to prevent accidental access to the injection needle and to reduce the risk of injury. In an autoinjector, the needle protection sleeve can also serve as a trigger element for triggering the product delivery process, wherein the needle protection sleeve is displaced relative to the housing in the proximal direction for this purpose. Alternatively, the triggering of the autoinjector can be achieved by actuating a trigger button of the autoinjector, wherein the needle protection sleeve serves at least as a visual protection before the autoinjector is used.

WO 2016/205963 describes an exemplary autoinjector comprising a housing with a longitudinal axis and a product container arranged axially fixedly in the housing. The autoinjector furthermore comprises a needle protection sleeve which is displaceable in a longitudinal direction between a proximal and a distal position and is coupled to a needle protection spring. A spiral or mainspring in which energy for the automatic delivery of product can be stored is connected via a first end to the housing and is connected via a second end in a rotationally fixed manner to a drive element in the form of a rotating threaded rod arranged coaxially with the longitudinal axis. The threaded rod engages via a thread in a propulsion member in the form of a propulsion sleeve, which propulsion member is not rotating in the housing and which propulsion sleeve moves the stopper of the product container at an approximately constant delivery speed in the distal direction during a displacement.

A mainspring as a drive is characterized by high forces and is thus suitable for a delay—free start of delivery, even with autoinjectors with a long storage period. A variable thread pitch of the threaded rod can compensate for a variable characteristic curve of the mainspring in order to ensure the most constant possible delivery force. This means that even large delivery volumes of 5 ml or more can be delivered evenly and continuously within a maximum of 60 seconds. With an extended delivery time of more than 10 s compared to conventional autoinjectors, the user should be able to ensure that the injection is proceeding as intended at all times in order to avoid inadvertently stopping the injection prematurely.

WO 2012/173554 shows a rotating display at the proximal end of an autoinjector, which is driven by a torsion spring during delivery. The display comprises a plurality of segments with different colors for signaling an initial state prior to the commencement of delivery and a final state after delivery has taken place. The segments are visible through at least one window at the proximal end of the autoinjector, the extension of which corresponds to the angular range of a segment.

CH 714527A2 discloses an injection device with a cap for removing a needle protection cap from a product container and a method for assembling an injection device, wherein the cap comprises an engagement element to cause removal of the needle protection cap from the product container when the cap is removed from the injection device.

US 2014/0330214 A1 shows a display in the form of a rotating disk and a plurality of window openings and wall panels at the proximal end of an autoinjector. The rotating disk has an alternating raised pattern for visual signaling and is driven by a torsion spring during delivery. The wall panels, which alternate with window openings, allow an interrupted view of the rotating disk or the structures rotating past.

EP 3241580 A1 shows an improved snap connection between the carpule holder and the device body. Reduced radial dimensions of the snap elements reduce the deformation of the carpule holder during final assembly, thus increasing safety against glass breakage of the carpule.

WO 2013/076246 A1 shows an autoinjector with a front and rear housing telescopically coupled in a first or second position, which coupling can be opened by the user in the second position to insert a new syringe.

WO 2013/153011 A1 shows a carpule holder which can be connected to a device housing by means of a snap connection. The snap connection has an improved force flow as the protrusions in the device housing engage in windows with rounded corners on the carpule holder.

WO 2016/055625 A1 shows an improved coupling of a carpule holder to the outer housing of an administration device. In this case, a first snap connection between the carpule holder and the outer housing is supplemented by a separate axially offset second connection between the carpule holder and the outer housing as well as by a third connection as an anti-twisting protection on a distally extending projection of the outer housing.

### SUMMARY

It is an object of the present disclosure to provide an improved, more cost-effective autoinjector of the type mentioned at the outset. The assembly of the autoinjector or its units and the insertion of a product container during final assembly shall be more reliable, and the operation and handling of the autoinjector shall be simpler and safer. The pre-assembly of units, which is separate in time and place from the final assembly of the autoinjector, as well as the storage, transport and supply of the same, shall be simpler and safer; in particular, the integrity of the units shall be maintained during transport and supply. It is a further object to create a display for an autoinjector of the type mentioned at the outset, which can visually display to the user the persistence or progression of a delivery in a wide variety of grip positions. The display shall be configured in such a way that even in unfavorable—in particular, unintended—grip positions of a user's hand on the autoinjector, the user can recognize the persistence of the delivery without having to change the grip position.

The objects are achieved by devices having the features of the present disclosure.

An autoinjector according to the present disclosure includes a one-piece or multi-part housing with a longitudinal axis and a pre-filled, ready-to-use syringe with a product container and an injection needle or cannula which is non-detachably fastened thereto. The ready-to-use syringe is axially non-displaceably received in the housing, where a tip of the injection needle projects by at least one piercing depth in the distal direction beyond a distal housing end. The autoinjector furthermore includes a torsion spring preloaded for the single delivery of a maximum content of the product container, a drive element, a propulsion element, and a needle protection sleeve. For delivering liquid from the product container through the injection needle, the torsion spring puts the drive element into rotation about the longitudinal axis, and the rotating drive element causes a linear movement of the propulsion element in order to displace a piston in the product container. When the autoinjector is pressed against a point of injection and the injection needle is caused to pierce the injection site as a result, the needle protection sleeve is moved in the proximal direction by an actuation stroke, thereby starting or allowing for a delivery of liquid. The actuation stroke of the needle protection sleeve corresponds here to at least the piercing depth of the injection needle.

The autoinjector finally includes a display for signaling the persistence of a delivery, having a display element, which is driven by the drive element, rotates about the longitudinal axis, and has an optical contrast pattern, and a window at the proximal end of the autoinjector, through which window the rotating contrast pattern is visible. The window is completely circumferential by 360° about the longitudinal axis, i.e., not interrupted by a frame or web parallel to the longitudinal axis, which creates a dead angle for the view on the display element. The window is constructed of a transparent and stable or load-bearing material. As long as even a small part of the window remains visible, i.e., even if the user clumsily covers the majority of the window with their hand holding the autoinjector, e.g., by supporting the proximal end with the heel of their hand, the rotating display element can still be recognized.

The contrast pattern is at most discretely rotationally symmetrical about the longitudinal axis, but not continuously rotationally symmetrical, so that a rotation of the contrast pattern can also be recognized. The contrast pattern includes at least one surface, zone or graphical element in a color or gray tone or brightness different from a background color, for instance in the form of graphical elements repeating in the direction of rotation, such as parallel lines, in particular parallel lines which extend in a manner inclined and/or curved relative to the longitudinal axis of the autoinjector. The contrast pattern can have at least one to approx. 60, in particular between 8 and 16, lines or surfaces or graphical elements. The contrast pattern contains in particular one of the physiologically easily perceptible colors, green or black.

For this purpose, the display element can be manufactured or configured as follows.

The contrast pattern can be applied to the base body of the display element using the pad printing process.

The contrast pattern can also be produced in different colors using 2-component injection molding. The two components may be divided in such a way that the first component, e.g. constructed of PBT, contains the functional elements such as the snap fasteners. This means that the functional elements are always created with the same material and/or in the same color. The visual, aesthetic elements such as the contrast pattern can be achieved with the 2nd component, e.g. constructed of colored ABS or PP, and vary in color.

The contrast pattern can also be created using a laser process—e.g. by means of a UV laser—by locally removing material from the display element on the visible surface, thereby creating a modified, in particular gray, structure. The surface of the display element can also be melted using a laser process and a suitable choice of material, as a result of which pigments become visible, or the plastics material foams up due to its ingredients, thereby modifying the optical properties in a contrasting manner. Alternatively, material can be removed locally and finely structured by laser or erosion on the previously polished visible surface of the injection mold, as a result of which structures—in particular, roof or cube structures—can be created, which are later transferred to the plastics material part during injection molding. Such structures can have a depth or raised surface of 0.01 to 0.5 mm. This is an advantageous design for reasons of sustainability, since, for the component, no different materials are mixed, no paint is applied to the material, and the material is not modified locally by laser marking.

The contrast pattern can also be created by means of openings in the visible surface of the display element. In this case, the openings allow a view into the dark-looking interior of the autoinjector, in particular onto the metal spiral torsion spring.

An autoinjector according to the present disclosure is suitable for delivering the amount of product contained in the product container during a delivery time of more than 10 s—for instance more than 20 s or 30 s—which is longer than that of known autoinjectors. As a result, even with product quantities of more than 2.25 ml and for instance at least 3 ml or 4 ml, a continuous or average delivery rate is relatively low, so that the inserted quantity of medication can be continuously absorbed or resorbed by the subcutaneous tissue. Therefore, maintaining a minimum holding time between the delivery end and moving the autoinjector away from the puncture site is less critical. Accordingly, no visual, acoustic, or tactile signaling is required to display the end of a holding time to the user.

In an embodiment, the rotating display element is driven directly or immediately—for instance, without gearing—by the drive element. The display element thus rotates at the same rotational speed as the drive element.

In a further embodiment, the display element carries out less than one revolution, for instance less than half a revolution or even less than a third of a revolution, per second during delivery. This ensures that the rotational movement of the contrast pattern can be easily followed by the eye and does not blur into an indeterminate, rotationally symmetrical pattern for the viewer.

In an embodiment, neither on the window nor on the adjoining housing is an optical marker for determining a relative rotational position of the contrast pattern provided. The display element does not display a start or end state, nor does it provide any relative information on the progress of the delivery; it is used exclusively to signal the progress or persistence of the delivery.

In an embodiment, the rotating display element and the housing or the window are configured to create a delivery sound, thereby additionally signaling to the user the persistence of the delivery process acoustically. For instance, the delivery sound is a continuous clicking sound, which is created by a grid on the window or on the housing and a radially or axially flexible engagement element engaging therein on the display element, or, conversely, by a rotating grid on the display element and a stationary engagement element. In this case, as with a purely optical movement indicator, a separate optical, acoustic, and/or tactile signaling of the delivery end, e.g., by a mechanically or electronically generated signal, can be omitted.

In an embodiment, the window has a first, cylindrical region parallel to the longitudinal axis, and a second region seamlessly adjoining the first in the proximal direction with a constantly or continuously decreasing diameter. The first region allows for a view of the display element from any lateral viewing direction perpendicular to the longitudinal axis, and the second region allows for a view of the display element in the distal viewing direction from behind, or from above when the autoinjector is held vertically. The second region is rounded or constricted in the proximal direction and can end in a third region perpendicular to the longitudinal axis. The display element can have a convex display surface following the course of the window or a cone-shaped display surface with a medium inclination with respect to the longitudinal axis. For instance, the window is rotationally symmetrical with respect to the longitudinal axis, i.e., with circular cross sections in sectional planes perpendicular to the longitudinal axis.

In an advantageous variant, the autoinjector includes a needle protection sleeve, which projects distally over the housing in the delivery state of the autoinjector and is preloaded in the distal direction by a needle protection spring. When the autoinjector is pressed against a point of injection, the needle protection sleeve carries out an actuation movement in the proximal direction and, when the autoinjector is removed from the point of injection, carries out a needle protection movement in the distal direction, in order to laterally surround and radially surround the injection needle with a sleeve-shaped, e.g. rotationally symmetrical, portion. The needle protection sleeve has, at one distal end, an annular flange or base as an enlarged support surface for contact with the tissue around the injection site. The flange is permanently connected to the sleeve-shaped portion and is for instance formed in one piece with it. A maximum diameter of the flange is greater than a maximum diameter of the sleeve-shaped portion.

The outer edge or the periphery of the flange may be adapted to the shape of a distal opening in the housing. In the inserted state, the flange thus forms a closure for the housing. The flange may also be configured to be concavely curved, so that the outer edge is further distal than the transition between the flange and the sleeve-shaped portion. Compared to a flange with a strictly flat, ring-shaped contact surface with an identical outer diameter, this, firstly, makes it less likely that the attached autoinjector will tilt, and, secondly, the concentration of the pressure load on the periphery of the flange furthest away from the puncture site results in less counter-pressure in the tissue and therefore less pain for the user.

Further, the autoinjector may include a threaded rod as the drive element and a propulsion sleeve with an internal thread as the propulsion element—alternatively, a drive sleeve with an internal thread as the drive element and a threaded rod as the propulsion element—where the propulsion element has a groove or a cam as an axial guide element for an exclusively linear propulsion movement in the housing. The autoinjector may be dimensioned for receiving a pre-filled, ready-to-use syringe including the product container and the injection needle, and may have a filling volume of at least 3 ml—or of at least 5 ml.

In summary and in other words, an autoinjector according to aspects of the present disclosure can be configured as follows:

Autoinjector including
a housing with a longitudinal axis L and for receiving a product container,
a propulsion member and a rotating drive element for moving the propulsion member in the longitudinal direction and for automatically delivering a liquid product contained in the product container through an injection needle,
a display for signaling the persistence of a delivery, including a rotating display element driven by the drive element and having an optical contrast pattern, and including a window at the proximal end of the autoinjector, through which window the rotating contrast pattern is visible, characterized in that the window is a window completely peripherally about the longitudinal axis.
   Autoinjector, where the rotating display element is driven directly by the drive element.

Autoinjector, where the display element carries out less than one, for instance less than half of one, revolution per second during a delivery.

Autoinjector, where no optical marking for determining a rotational position of the contrast pattern is provided.

Autoinjector, where the rotating display element is configured to create a delivery sound.

Autoinjector, where the window has a first, cylindrical region parallel to the longitudinal axis, and a second region, seamlessly adjoining the first in the proximal direction, with a constantly decreasing diameter.

Autoinjector, further including
a needle protection sleeve, which projects distally over the housing in the delivery state of the autoinjector, where, when the autoinjector is pressed against an injection site, the needle protection sleeve carries out an actuation movement in the proximal direction and, when the autoinjector is removed from the point of injection, carries out a needle protection movement in the distal direction in order to laterally surround the injection needle with a sleeve-shaped portion, where the needle protection sleeve has an annular flange at a distal end for contact with the injection site, with a maximum diameter that is greater than the maximum diameter of the sleeve-shaped portion.

Autoinjector, where an outer edge of the flange is adapted to a distal opening in the housing.

Autoinjector, where the flange is concavely curved.

Autoinjector where the drive element is a threaded rod, and the propulsion element is a propulsion sleeve with an axial guide element for an exclusively linear propulsion movement in the housing.

Autoinjector further having a pre-filled ready-to-use syringe including the product container and the injection needle, and having a filling volume of at least 3 ml, or of at least 5 ml.

Further embodiments of the invention, for an improved autoinjector, are described below.

In an embodiment, an autoinjector includes
a drive device including a sleeve-shaped first housing part including a drive assembly, including a spring coil with a mainspring, a drive element, in particular a threaded rod, a propulsion element, in particular a piston rod with an internal thread suitable for automatically propelling the stopper
a product container receiving device having a sleeve-shaped second housing part defining a longitudinal axis which extends from distal to proximal, including a syringe holder configured to receive a pre-filled ready-to-use syringe having a cylindrical product container which tapers at its distal end over a shoulder with a needle protection cap of a needle and a stopper which can be moved along the longitudinal axis by the drive assembly for delivering a medication from the product container, a device cap in which the needle protection cap can be received, where the first housing part and the second housing part can be connected coaxially in a joining direction, in that the connection-side end portion on one of the two housing parts is stepped in such a way that it can be inserted into the other of the two housing parts, where a peripheral axially acting gap guide is formed between the two housing parts in the region of the end portion, where, in the region of the end portion, a first snap-in connection and a second or middle and a third or last, final snap-in connection are provided, which can be snapped into place one after the other in the joining direction and where the first of the snap-in connections can be released non-destructively against the joining direction and at least the third or final snap-in connection is locked, in particular by positive locking. This means that the housing parts can be connected one after the other in the joining direction in defined positions or defined linear movement steps or can be separated against the joining direction, which creates advantages for storage, transport and further processing on a machine. The locking by at least the third or final snap-in connection cannot be opened non-destructively, which may provide the operating and manipulation safety of the autoinjector. In this case, the autoinjector having a locked third or final snap-in connection achieves its configuration and/or dimension for delivery or use.

The autoinjector is further developed in that the first housing part and the second housing part can be mutually connected by a linear movement parallel to the longitudinal axis in the joining direction or can be released counter to the joining direction. The, in particular all the, insertion movements or the movements for joining the housing parts in and releasing the housing parts counter to the joining direction are aligned and in particular run exclusively in or parallel to the longitudinal axis. This does not include more complex movements of the housing parts which are caused, for example, by bayonet, screw or threaded closures. This allows for cost-effective and precise handling of the parts on assembly machines and, thanks to the connectability and detachability, the parts can be stored or transported together in a defined position prior to final assembly, i.e., before inserting a product container. The interior of the autoinjector pre-assembled in this way is protected. The parts connected in this way can be stored and transported as a unit in a module carrier in a space-saving and safe manner. The parts or assemblies connected in this way can be easily taken over by a machine, separated in a defined position for the insertion of the ready-to-use syringe, and then reconnected and ultimately fully assembled.

The autoinjector is further developed in that a first, second and third snap-in position of the first housing part relative to the second housing part along the longitudinal axis is defined by the first snap-in connection and the second and third snap-in connections. This allows for simplified automation and positioning on the assembly machine.

The autoinjector is further developed in that the first, second and third snap-in connections are formed from cams and grooves formed on the first and second housing parts, in the interior of the gap guide. This increases protection against unauthorized opening and tampering.

The autoinjector is further developed in that one of the housing parts, with its radial step introducing the tapered end portion, can be completely abutted all the way around the connection-side end of the other housing part and thus the two housing parts together form a seamless outer surface, which makes handling of the autoinjector safer, where the radial step can be straight or continuously wavy or discontinuously offset in the peripheral direction. The latter may provide the anti-twisting protection and can support the rotational positioning of the housing parts. In addition, the radial step can rotate at a right angle or inclined to the axis. Alternatively, the step formed on one housing part can be abutted in a peripherally interrupted manner at the connection-side end of the other housing part.

The autoinjector is further developed in that one of the housing parts, with its radial step introducing the end portion, abuts peripherally against the connection-side end of the other housing part when the third or final of the snap-in connections is locked in a form-fitting manner. By keeping the joint thus formed closed by the locking mechanism, a better contamination tightness and security against manipulation is achieved.

The autoinjector is further developed in that the two housing parts have longitudinal guides in the region of the end portion and/or form a non-circular cross section of the gap guide. This results in an anti-twisting protection and a defined mutual assembly position of the two housing parts. For instance, the gap guide in the cross section is not circularly centrally symmetrical, in particular 2-fold or 4-fold centrally symmetrical, i.e. possible assembly positions arise every 180° or 90°. Further, axially extending structures of the snap-in connections may be mutually aligned on two housing parts with respect to the longitudinal axis of the autoinjector and thus form longitudinal guides.

The autoinjector is further developed in that one of the housing parts has at least one assembly opening at its end portion, which is accessible from the outside at least in the first snap-in position and is completely covered by the other of the housing parts in at least the third or final snap-in position. This means that an assembly tool can be inserted and removed for final assembly before the final joining, and the opening required for this is then completely closed and is no longer visible from the outside. The assembly tool extending into the interior of the housing part can be used to hold a component that is movably mounted inside the housing and/or subjected to force or torque at a defined location during an assembly movement and to release it again afterwards. For example, this can ensure that the component is snapped into place or anchored.

The autoinjector is further developed in such a way that the two housing parts can assume exactly three snap-in positions along the longitudinal axis.

The autoinjector is further developed in that the two housing parts are fastened to one another in the third or final snap-in position alternatively or in addition to the snap-in connection by gluing or welding. This increases the stability and precision of the connection and ensures the tightness of the butt joint between the two housing parts.

The autoinjector is further developed in that it further includes a syringe adapter, which can be abutted with its distal end against the proximal end of the ready-to-use syringe, where the proximal end of the syringe adapter is formed by a resilient element which is operatively connected to the first housing part. This means that the syringe is held in the syringe holder with no play in the distal direction and is force-loaded, which reduces the risk of glass breakage.

The autoinjector is further developed in that the resilient element of the syringe adapter, to which the support surface is connected, is pre-tensioned or further tensioned by the second joining step or by the movement into the third or final snap-in position. This simplifies the final assembly process.

The autoinjector is further developed in that the propulsion element can be screwed onto the drive element in a desired starting position. Thus, in the fully assembled autoinjector, the clearance between the piston in the ready-to-use syringe and the propulsion element can be eliminated and/or the starting position of the propulsion element can be adapted to the piston position of a partially filled ready-to-use syringe.

In addition, the illustrated invention includes a method for the final assembly of an autoinjector which defines a longitudinal axis, including several of the steps of:
providing a product container receiving device including a first housing part
providing a drive device including a second housing part
joining the housing parts in the joining direction into a first snap-in position defined by a first snap-in connection
inserting, in particular laterally inserting, an assembly tool through an assembly opening on one of the housing parts when they are in the first snap-in position
joining the housing parts in the joining direction into a second snap-in position which is held by a second snap-in connection of the housing parts
removing the assembly tool from the assembly opening on one of the housing parts when they are in the second snap-in position
joining the housing parts in the joining direction into a third snap-in position which is held and locked by a third snap-in connection of the housing parts
completely covering the assembly opening on one of the housing parts by the other of the housing parts when they are in the third or final snap-in position
setting or preparing an axial starting position for a propulsion element by screwing it onto a drive element. Thus, in the fully assembled autoinjector, the clearance between the piston in the ready-to-use syringe and the propulsion element can be eliminated and/or the starting position of the propulsion element can be variably adapted to the piston position of a partially filled ready-to-use syringe.
positioning the product container receiving device and the drive device coaxially to the longitudinal axis and joining the housing parts in the joining direction into a snap-in position which is held by a snap-in connection of the housing parts that can be released in and counter to the joining direction
storing and/or transporting the product container receiving device and the drive device connected together in the snap-in position of the housing parts
providing a ready-to-use syringe
releasing the snap-in connection of the housing parts counter to the joining direction and separating the drive device from the product container receiving device
inserting the ready-to-use syringe coaxially or axially parallel to the longitudinal axis into the product container receiving device
holding and/or positioning a component that is movable inside one of the housing parts by means of the assembly tool inserted through the assembly opening
clamping a resilient syringe adapter when joining the housing parts from the second to the third or final snap-in position.

Alternatively or additionally, the method for the final assembly of an autoinjector which defines a longitudinal axis may include several of the following steps:
providing a product container receiving device including a first housing part
providing a drive device including a second housing part
positioning the product container receiving device and the drive device coaxially to the longitudinal axis and joining the housing parts by a linear movement into a first snap-in position of at least three snap-in positions along the longitudinal axis, which first snap-in position is held by a first snap-in connection of the housing parts that can be released non-destructively in and counter to the joining direction
storing and/or transporting the product container receiving device and the drive device connected together in the first snap-in position of the housing parts. This ensures the integrity and mutual position of the parts of the device during storage or transport and allows subsequent processing on an assembly machine to be simple and precise.
providing a ready-to-use syringe
releasing the snap-in connection of the housing parts and separating the drive device from the product container receiving device
inserting the ready-to-use syringe coaxially or axially parallel to the longitudinal axis into the product container receiving device
joining the housing parts by a linear movement along the longitudinal axis into the first snap-in position
joining the housing parts by a linear movement along the longitudinal axis into a second snap-in position which is held by a second snap-in connection of the housing parts
joining the housing parts by a linear movement along the longitudinal axis into a third snap-in position which is held and locked by a third snap-in connection of the housing parts
releasing an assembly opening on one of the housing parts when they are in the first snap-in position
holding and/or positioning a component movable inside one of the housing parts by means of an assembly tool that can be inserted through the released assembly opening, in particular an assembly tool that can be inserted laterally
removing the assembly tool from the assembly opening on one of the housing parts before or when they are in the second snap-in position
completely covering the assembly opening by the other of the housing parts when they are in the third or final snap-in position
clamping a resilient syringe adapter during joining or through the joining movement of the housing parts from the second to the third or final snap-in position. In this case, the syringe adapter is arranged in such a way that the resilient elements at least partially follow the joining movement.

### BRIEF DESCRIPTION OF THE DRAWINGS

Embodiments of the present disclosure are described below in connection with the appended figures. These embodiments are intended to show basic possibilities and are in no way to be interpreted as limiting. In the drawings:

FIG. 1 shows the components of an autoinjector,

FIGS. 2A and 2B show longitudinal cross-sections through the autoinjector of FIG. 1 rotated by 90° against each other before injection,

FIGS. 3A and 3B show isometric views of a display element,

FIG. 4 is a view of a distal end of the needle protection sleeve,

FIGS. 5A and 5B show views of a second embodiment of an autoinjector rotated by 90° against each other,

FIG. 6 shows the components of the autoinjector or the assemblies from FIGS. 5A and 5B,

FIGS. 7A and 7B show two longitudinal cross-sections of the autoinjector from FIGS. 5A and 5B in the delivery state, rotated by 90° against one another,

FIGS. 8A and 8B show two longitudinal cross-sections of the autoinjector from FIGS. 5A and 5B rotated by 90° against one another in the trigger lock state,

FIGS. 9A and 9B show two longitudinal cross-sections of the autoinjector from FIGS. 5A and 5B rotated by 90° against one another, in the removed state of the device cap including the needle protection cap,

FIGS. 10A and 10B show two longitudinal cross-sections of the autoinjector from FIGS. 5A and 5B rotated by 90° against one another in the inserted state,

FIGS. 11A and 11C show two longitudinal cross-sections of the autoinjector from FIGS. 5A and 5B rotated by 90° against one another in the drive release state,

FIG. 11B is a view of a partial cross-section of the coupling sleeve and the spring coil of the autoinjector from FIGS. 5A and 5B in the drive release state,

FIGS. 12A and 12D show two longitudinal cross-sections of the autoinjector from FIGS. 5A and 5B rotated by 90° against one another, in the delivering state,

FIG. 12B is a proximal view of the spring coil with the spiral spring of the autoinjector from FIGS. 5A and 5B,

FIG. 12C is a cross section from a distal view of the display element with display window of the autoinjector from FIGS. 5A and 5B,

FIGS. 13A and 13B show two longitudinal cross-sections of the autoinjector from FIGS. 5A and 5B rotated by 90° against one another in the delivering state,

FIGS. 14A and 14B show two longitudinal cross-sections of the autoinjector from FIGS. 5A and 5B rotated 90° against one another in the needle protection lock activated state,

FIGS. 15A and 15C show two longitudinal cross-sections of the autoinjector from FIGS. 5A and 5B rotated by 90° against one another in the delivery broken off state,

FIG. 15B is a view of a partial section of the coupling sleeve and the spring coil of the autoinjector from FIGS. 5A and 5B in the delivering state,

FIGS. 16A and 16B are a plan view and a longitudinal cross-section of the autoinjector from FIGS. 5A and 5B in the pre-snapped state,

FIGS. 17A and 17B show two longitudinal cross-sections of the product container receiving device with insert pre-assembled, rotated by 90° against one another,

FIGS. 18A and 18B show two longitudinal cross-sections of the product container receiving device with pre-assembled ready-to-use syringe rotated by 90° against one another,

FIGS. 19A and 19B show two longitudinal cross-sections of the product container receiving device with insert and fully assembled ready-to-use syringe, rotated by 90° against one another,

FIG. 20A is a longitudinal cross-section of the autoinjector from FIGS. 5A and 5B after the first joining step,

FIG. 20B is a longitudinal cross-section of the autoinjector from FIGS. 5A and 5B after the second final joining step,

FIG. 21A shows a longitudinal cross-section of the product container receiving device for a piercing depth by way of example of 8 mm in the delivery state,

FIG. 21B shows a longitudinal cross-section of the product container receiving device for a piercing depth by way of example of 8 mm in the inserted state,

FIG. 22A shows a longitudinal cross-section through the distal end of the product container receiving device for a piercing depth by way of example of 5 mm in the delivery state,

FIG. 22B shows a longitudinal cross-section through the distal end of the product container receiving device for a piercing depth by way of example of 5 mm in the inserted state,

FIG. 23A shows a longitudinal cross-section of the drive device as well as a view of the mainspring and the display window,

FIG. 23B shows a longitudinal cross-section through the proximal end of the drive device as well as a view of a variant of the mainspring and the display window,

FIG. 24A shows a longitudinal cross-section of the autoinjector from FIGS. 5A and 5B with a fully filled ready-to-use syringe,

FIG. 24B shows a longitudinal cross-section of the autoinjector from FIGS. 5A and 5B with partially filled ready-to-use syringe, and

FIGS. 25A and 25B show two views of a variant of the autoinjector from FIGS. 5A and 5B with a trigger sleeve.

### DETAILED DESCRIPTION

### Definitions

The term “product,” “medicament,” or “medical substance” in the present context includes any flowable medical formulation which is suitable for controlled administration by means of a cannula or hollow needle in subcutaneous or intramuscular tissue, for example a liquid, a solution, a gel, or a fine suspension containing one or more medical active ingredients. A medication can thus be a composition with a single active ingredient or a premixed or co-formulated composition with a plurality of active ingredients from a single container. The term includes in particular drugs, such as peptides (e.g., insulins, insulin-containing medicaments, GLP-1-containing preparations as well as derived or analogous preparations), proteins and hormones, biologically obtained or active ingredients, active ingredients based on hormones or genes, nutrient formulations, enzymes, and other substances both in solid (suspended) or liquid form. The term also includes polysaccharides, vaccines, DNA or RNA or oligonucleotides, antibodies or parts of antibodies as well as suitable base substances, excipients, and carrier substances.

The term “distal” refers to a side or direction directed toward the front, piercing-side end of the administration device or toward the tip of the injection needle. In contrast, the term “proximal” refers to a side or direction directed toward the rear end of the administration device that is opposite the piercing-side end.

In the present description, the term “injector” is understood to mean a device with which the injection needle is removed from the tissue after a controlled amount of the medical substance has been delivered. In contrast to an infusion system, the injection needle of an injector thus does not remain in the tissue for a longer period of several hours.

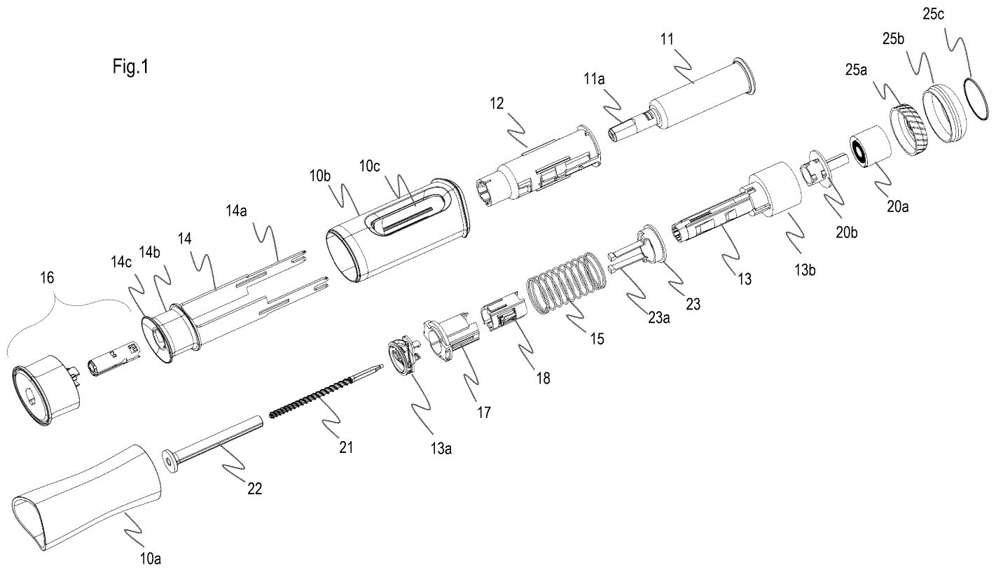

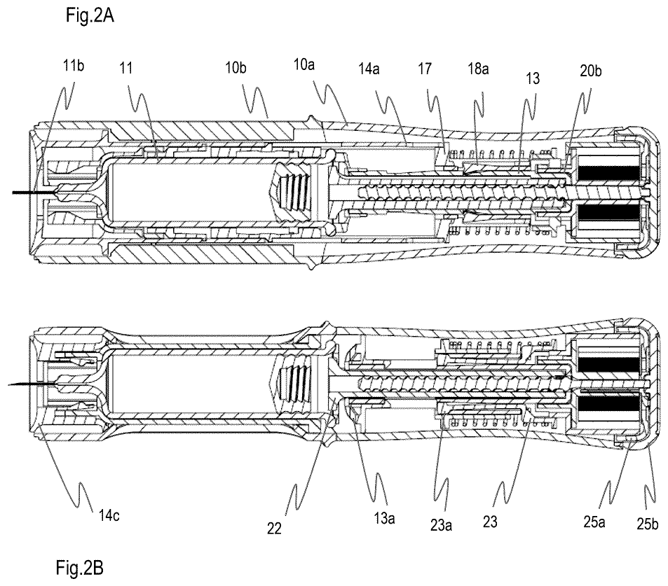

Turning to the Figures, FIG. 1 is an exploded view of the components of an autoinjector according to the invention in a first embodiment, and FIGS. 2A and 2B show two longitudinal cross-sections through the autoinjector according to FIG. 1 rotated by 90° to one another about the longitudinal axis in the inserted state and ready for delivering.

The autoinjector 1 has a sleeve-shaped, elongated housing with a longitudinal axis L and comprising a distal housing part 10b and a proximal housing part 10a in the form of a handle that is non-detachably snap-fitted therewith. A product container in the form of a ready-to-use syringe 11 with an injection needle which is non-detachably fastened to the product container is held in a syringe holder 12, wherein the syringe holder is received in the housing in an axially and rotationally fixed manner. The ready-to-use syringe 11 is pressed in a distal direction into engagement with a shoulder of the syringe holder 12 by a holding spring portion 13a fixedly anchored in the housing part 10a. In relation to the housing part 10b, the ready-to-use syringe 11 is arranged in such a way that the tip of the injection needle projects beyond the distal end of a proximal intermediate position of the needle protection sleeve 14 by a length corresponding to the subcutaneous or intramuscular piercing depth and is at least laterally protected or covered by a needle protection sleeve 14 before and after the injection. When the injection needle is inserted into the injection site along the longitudinal axis L, the needle protection sleeve 14 is pushed in the proximal direction by an actuation stroke and against the force of a needle protection spring 15, and thereby triggers a product delivery. For this purpose, the needle protection sleeve comprises two sleeve arms 14a, which are arranged offset or rotated by 90° about the longitudinal axis L with respect to two recesses 10c of the housing designated as viewing windows. After the injection has taken place, the needle protection sleeve 14 can be displaced relative to the housing part 10b from the actuated position along the longitudinal axis L in the distal direction to a needle protection position and can be blocked there against being pushed back again. The needle protection sleeve comprises a sleeve-shaped or hollow-cylindrical portion 14b and a flange 14c at the distal end. The cross section of the hollow-cylindrical portion 14b is oval, as is the outer circumference of the flange, so that, in the present case, the flange has a constant width or radial extension.

A spring assembly comprises a spiral spring 20a and a spring coil 20b. The outer end of the spiral spring 20a is non-rotatably fastened to a spring sleeve 13b as part of a mechanism holder 13 fixedly anchored in the housing. The inner end of the spiral spring 20a is connected to the spring coil 20b in a rotationally fixed manner. The spring coil 20b comprises a spring shaft and a distal spring flange. The spiral spring 20a or the spring coil 20b puts a drive element 21 into a rotational movement and a propulsion element 22 into a preferably purely axial propulsion movement. For this purpose, a threaded element engages in a thread extending over the delivery stroke and having a variable thread pitch.

The ready-to-use syringe 11 comprises a cylindrical syringe body as a product container, at the distal end of which a hollow injection needle is fixedly connected to a syringe shoulder. The injection needle of the ready-to-use syringe is covered by a needle protection cap 11a, which is configured as a so-called rigid needle shield (RNS) and comprises a rubber-elastic needle protection element and a sheath constructed of hard plastics material. The needle protection cap protects the injection needle against mechanical effects and contamination, and keeps the injection needle and the product sterile. At the distal end of the autoinjector, in the delivery state thereof, a two-part device cap or pull-off cap 16 is arranged, which is axially pulled off and/or twisted off and completely removed along with the needle protection cap 11a before the autoinjector is used.

A switching sleeve 17 is arranged in a form-fitting manner with a proximal end of the sleeve arms 14a of the needle protection sleeve 14 and with a distal end of the needle protection spring 15 and is at least partially surrounded by the latter. The switching sleeve 17 is preferably snap-fitted with the proximal end of the sleeve arms of the needle protection sleeve 14. The movement of the switching sleeve 17 in the distal direction is delimited by the holding spring portion 13a, which in turn is snap-fitted together with the mechanism holder 13 after the switching sleeve 17 has been assembled. A locking sleeve 18 is arranged within and coaxially with the switching sleeve 17 and is coupled to the switching sleeve 17 via a saw tooth-shaped locking member 18a, which is resiliently attached to an arm pointing in the distal direction, in such a way that an actuation movement of the needle protection sleeve 14 and the switching sleeve 17 also moves the locking sleeve 18 proximally. By means of an additional proximal locking stroke of the locking sleeve 18 relative to the switching sleeve 17 into a proximal end position, the locking member 18a is reliably released by the switching sleeve 17 for movement inward. Due to the spring effect of the arm, the locking member 18a engages behind a proximally directed edge of the autoinjector or latches into an axially fixed recess of the autoinjector and thus locks the locking sleeve 18 against a distal movement. When the autoinjector is removed from the puncture site, the switching sleeve 17 is pushed by the needle protection spring 15 in the distal direction over the locking member 18a, whereupon, as a result of the spring effect of the arm, the locking member engages behind a proximally directed edge of the switching sleeve 17 in a locking position and locks or blocks the switching sleeve and the needle protection sleeve against renewed movement in the proximal direction.

A coupling sleeve 23 with two holding cams 23a is coupled to the spring coil 20b via coupling elements. Before delivery, the holding cams 23a engage in recesses of the axially fixed mechanism holder 13 and are prevented from moving outwards by an inner circumference of the locking sleeve 18, as a result of which the coupling sleeve 23 also cannot move axially. When the delivery is triggered, the locking sleeve 18 is moved away from the position of the recesses by a proximal movement of the needle protection sleeve 14 so that the holding cams 23a can detach radially and the coupling sleeve 23 is released. The latter moves in the proximal direction and releases the spring coil 20b for rotation, as described in detail in WO 2022/069617 A1. At the proximal end of the autoinjector is the display with a rotating display element 25a with a contrast pattern in the form of parallel stripes, a peripheral transparent window 25b, and a proximal, non-transparent closure 25c.

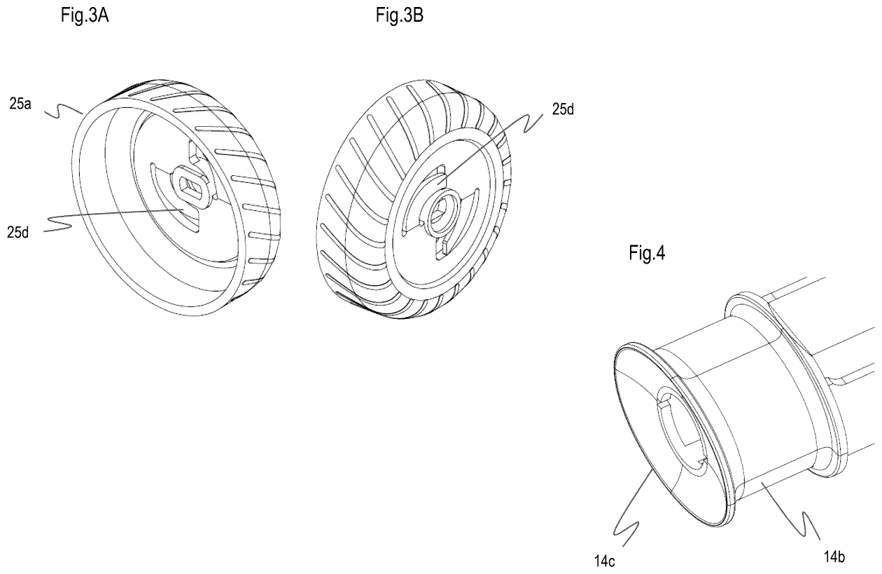

FIGS. 3A and 3B show two isometric views of the display element 25a with an engagement element 25d for engagement with a non-rotating grid on the inner side of the window 25b or the closure 25d. The engagement element 25d is spring-mounted and creates a number of clicking sounds corresponding to the snap-in grid when the display element rotates during delivery. The display element 25a has a shape that follows that of the window 25b or is adapted to it, with a first, distal region and a second region adjoining it in the proximal direction. The first region is cylindrical, parallel to the longitudinal axis, while the second region has a steadily decreasing diameter.

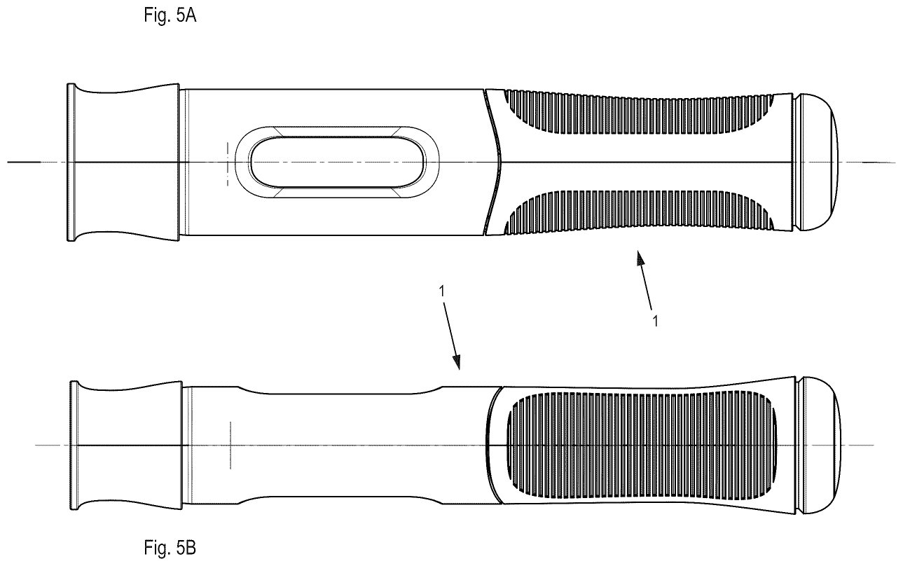

FIG. 4 shows the distal end of the needle protection sleeve with the sleeve-like portion 14b and the flange 14c. The shape of the flange is clearly visible with the concave curved contact surface, the outer, oval edge of which projects distally from the inner edge of the contact surface at the transition to the axis-parallel portion.

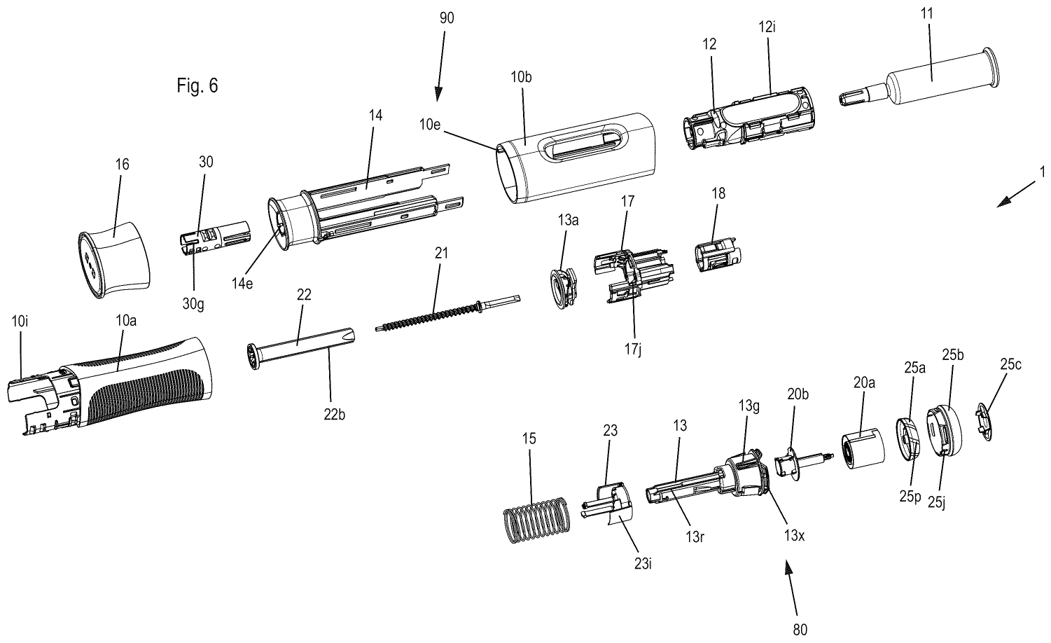

FIGS. 5A and 5B show two plan views rotated by 90° relative to one another of a second embodiment of an autoinjector 1 according to the invention.

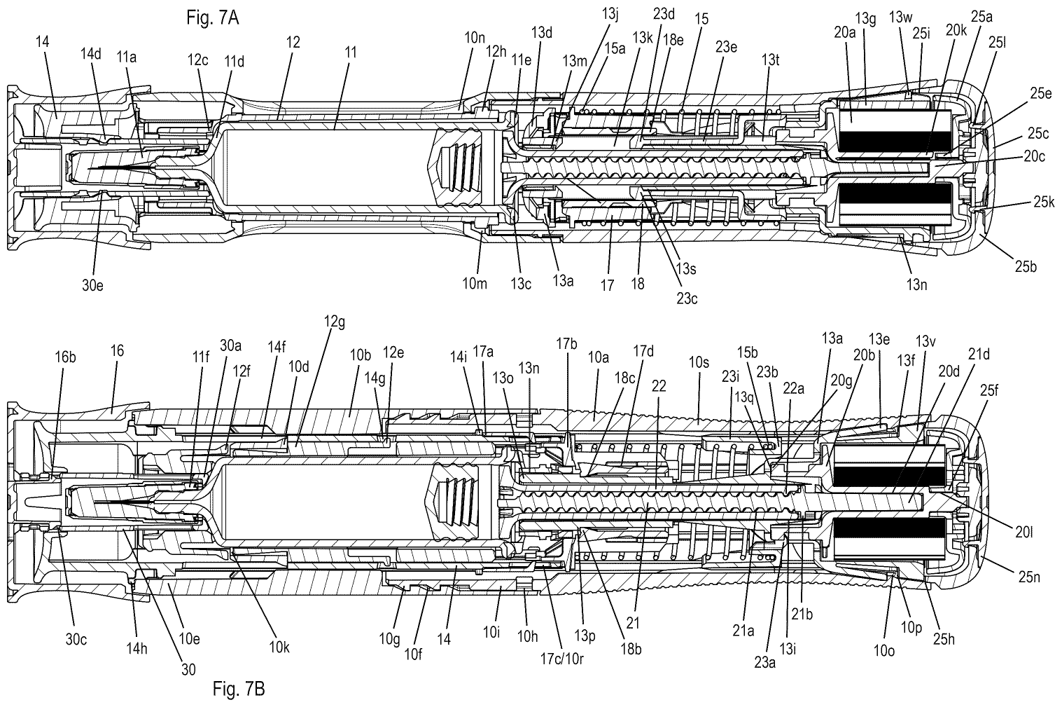

FIG. 6 is an exploded view of the components of a second embodiment of the improved autoinjector of FIGS. 5A and 5B. The autoinjector 1 has, as assemblies, a product container receiving device 90, a ready-to-use syringe 11, and a drive device 80. In this case, the product container receiving device 90 has the following components: a device cap 16, an insert 30, a needle protection sleeve 14, a syringe housing part 10b and a syringe holder 12. The drive device 80 has the following components: a drive housing part 10a, a propulsion element 22, a drive element 21, a syringe adapter 13a, a switching sleeve 17, a locking sleeve 18, a needle protection spring 15, a coupling sleeve 23, a mechanism holder 13, a spring coil 20b, a spiral or mainspring 20a, a display element 25a, a display window 25b and a display cap 25c. The autoinjector 1 can be fully assembled from the two pre-assembled assemblies 80, 90, ready for storage and/or use, by inserting the ready-to-use syringe 11 into the product container receiving device 90 and then firmly connecting the drive device 80 to the product container receiving device 90, in particular snapping it together.

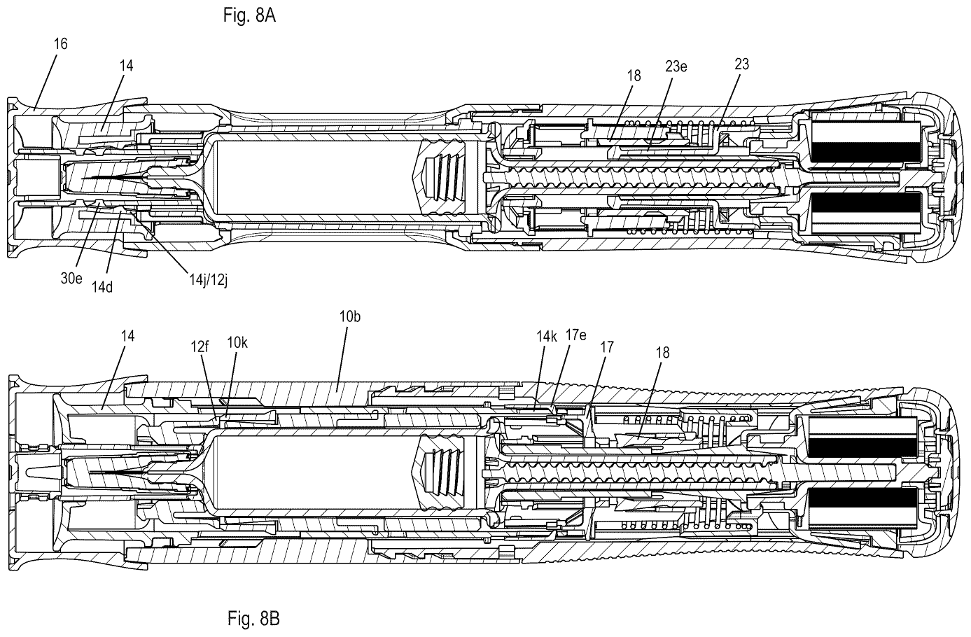

FIGS. 7A and 7B (see also FIG. 6, 11B, 12B) shows two longitudinal cross-sections of the autoinjector from FIGS. 5A and 5B, rotated by 90° relative to one another, in the state of delivery or before use. The device cap 16 is non-releasably positioned axially relative to the insert 30 by the holding cams 16b, which engage in the correspondingly arranged holding openings 30c of the insert 30. The rotational positioning of the device cap 16 is achieved by its anti-twisting ribs 16a (inside the device cap, not shown), which are in engagement with the anti-twisting grooves 30g of the insert 30 arranged correspondingly thereto, as well as the anti-twisting grooves 14e of the needle protection sleeve 14.

For positioning the insert 30, the latter has second holding openings 30e into which the correspondingly arranged holding catches 14d of the needle protection sleeve 14 engage, which can be released with the application of force. In addition, the holding cams of the holding arms 30a arranged at the proximal end of the insert 30 are in engagement with the shoulder 11d of the needle protection cap 11a of the ready-to-use syringe 11.

The needle protection sleeve 14 is positioned by its holding openings 14f, into which the correspondingly arranged holding cams 10d of the syringe housing part 10b engage, and is secured against axial displacement in the distal direction. The needle protection sleeve 14 also has further holding openings 14g, into which the holding cams 12e of the syringe holder 12 engage, which are arranged correspondingly thereto and can be released with the application of force. The needle protection spring 15 exerts with its distal spring end 15a a defined preload force via the switching sleeve 17 onto the needle protection sleeve 14, thereby defining its axial positioning. The rotational positioning of the needle protection sleeve 14 is achieved by its anti-twisting grooves 14h, into which the anti-twisting ribs 10e of the syringe housing part 10b, arranged correspondingly thereto, engage.

The syringe housing part 10b is considered together with the drive housing part 10a as a fixed part or, for information in this description, as a reference housing. It is axially and inseparably connected to the drive housing part 10a by its double snap groove 10f, the distal and proximal grooves of which are in engagement with the correspondingly arranged double holding cams 10g of the drive housing part 10a. The rotational positioning of the syringe housing part 10b is achieved by its anti-twisting ribs 10h, which are in contact with the correspondingly arranged anti-twisting surfaces 10i of the drive housing part 10a.

The syringe holder 12 is axially positioned by its holding catches 12f and by its support surfaces 12g, which are in contact with the correspondingly arranged support surfaces 10k, 101 of the syringe housing part 10b. The rotational positioning of the syringe holder 12 is achieved by its anti-twisting ribs 12h, which are in engagement with the anti-twisting grooves 10m of the syringe housing part 10b arranged correspondingly thereto. In addition, the rotational positioning of the syringe holder 12 is ensured by its anti-twisting ribs 12i, which are in contact with the correspondingly configured viewing window edge 10n of the syringe housing part 10b.

The ready-to-use syringe 11 is axially positioned in the distal direction by its support surface 11d, which is in contact with the correspondingly arranged support edges 12c of the syringe holder 12, and in the proximal direction by its support surface Ile, which is in contact with the correspondingly arranged support surface 13c of the syringe adapter 13a, wherein the flexible elements 13d of the syringe adapter 13a, to which the support surface 13c is connected, exert a defined preload on the ready-to-use syringe 11. The ready-to-use syringe 11 is rotatively free.

The syringe housing part 10b is considered together with the drive housing part 10a as a fixed part of the reference housing.

The mechanism holder 13 is axially positioned by its holding catches 13e and the support surfaces 13f, which are in contact with the correspondingly configured holding cams 100 and the support surfaces 10p of the drive housing part 10a. The rotational positioning is ensured by the anti-twisting ribs 13g, which are in engagement with the corresponding anti-twisting grooves 10q (not shown) of the drive housing part 10a.

The axial position of the propulsion element 22 is defined by its internal threaded portion 22a, which is in engagement with the correspondingly configured external threaded portion 21a of the drive element 21. The rotational positioning of the propulsion element 22 is achieved by its axially extending anti-twisting rib 22b, which is guided in the anti-twisting groove 13h (not shown) of the mechanism holder 13 arranged corresponding thereto.

The drive element 21 is axially positioned by its peripheral shoulder 21b, at the distal end of which the correspondingly arranged holding catches 13i of the mechanism holder 13 engage, and by its proximal support surface 21c, which is in contact with the correspondingly configured support surface 20c of the spring coil 20b. The rotational positioning of the drive element 21 is achieved by its anti-twisting surfaces 21d, which are in contact with the corresponding anti-twisting surfaces 20d of the spring coil 20b.

The syringe adapter 13a is axially positioned by its holding catches 13j, which are in contact with the correspondingly arranged holding openings 13k, and by its support surfaces 131, which are in contact with the correspondingly configured support surfaces 13m of the mechanism holder 13. The rotational positioning of the syringe adapter 13a is achieved by its anti-twisting grooves 13n, which engage with the correspondingly arranged anti-twisting ribs 130 of the mechanism holder 13.

The switching sleeve 17 rests at its distal end with its support surfaces 17a, which are in contact with the corresponding contact surfaces 14i of the needle protection sleeve 14, and is preloaded by the distal spring end 15a of the needle protection spring 15, which rests on the corresponding spring support 17b of the switching sleeve 17, by the defined spring force of the needle protection spring 15. The rotational positioning of the switching sleeve 17 is achieved by its guide surfaces 17c, which are in engagement with the correspondingly configured guide ribs 10r of the drive housing part 10a.

The locking sleeve 18 is axially positioned within the switching sleeve 17 with a defined clearance. The locking sleeve 18 is delimited in the distal direction by its inwardly directed holding cams 18b, by contact with the correspondingly arranged support surfaces 13p of the mechanism holder 13. In the proximal direction, the locking sleeve 18 is delimited by the outwardly directed holding cams 18c, by contact with the correspondingly configured holding openings 17d of the switching sleeve 17. The rotational positioning of the locking sleeve 18 is achieved by its inwardly directed guide surfaces 18d (not shown), which are in engagement with the correspondingly configured guide surfaces 13r of the mechanism holder 13, as well as via a positive locking with the switching sleeve 17.

The needle protection spring 15 is positioned by its distal spring end 15a, which rests on the correspondingly configured spring support 17b of the switching sleeve 17, and its proximal spring end 15b, which rests on the correspondingly configured spring support 23b of the coupling sleeve 23, wherein the needle protection spring 15 exerts a defined preload between the switching sleeve 17 and the coupling sleeve 23 through its spring force. In addition, the needle protection spring 15 is guided with its outer surface through the internal guide ribs of the drive housing part 10a and is laterally supported by peripheral aprons 23i.

The coupling sleeve 23 rests with its inwardly directed holding cams 23c, which are in contact with the correspondingly arranged holding surfaces 13s of the mechanism holder 13, wherein the outwardly directed contact surfaces 23d, which are in contact with the correspondingly arranged contact surfaces 18e of the locking sleeve 18, prevent the holding arms 23e of the coupling sleeve 23 from deflecting outwards. The axial position of the coupling sleeve 23 is defined by the defined preload of the spring force of the needle protection spring 15, which rests with its proximal spring end 15b on the correspondingly configured spring support 23b. The torque of the spiral spring 20a applied to the spring coil 20b is blocked by the inclined contact surfaces 20f of the coupling cams 20e, which are in contact with the correspondingly arranged contact surfaces 23f of the coupling cams 23a of the coupling sleeve 23, wherein the angle of inclination of the contact surfaces 20f, 23f has been defined such that the applied torque acts partly as an axial force on the coupling sleeve 23, whereby the coupling sleeve 23 is preloaded in the proximal direction. The rotational positioning of the coupling sleeve 23 is achieved by the holding arms 23e, which are in engagement with the correspondingly configured guide openings 13t of the mechanism holder 13, as well as by the contact of the coupling cams 20e with the coupling cams 23a.

The spring coil 20b rests with its support surface 20g attached to the distal end on the correspondingly configured support surface 13q of the mechanism holder 13. The torque of the spiral spring 20a applied to the spring coil 20b is blocked by the inclined contact surfaces 20f of the coupling cams 20e, which are in contact with the correspondingly arranged contact surfaces 23f of the coupling cams 23a of the coupling sleeve 23, wherein the angle of inclination of the contact surfaces 20f, 23f has been defined such that the applied torque acts partly as an axial force on the spring coil 20b, whereby the spring coil 20b is pressed against the mechanism holder 13 with a slight preload force and is axially fixed.

The spiral spring 20a is in engagement with its outer spring end 20h in the correspondingly configured spring receptacle 13u of the mechanism holder 13 and with its inner spring end 20i in engagement with the correspondingly configured spring receptacle 20j of the spring coil 20b. The spiral spring 20a is positioned axially between the spring coil 20b and the display element 25a. The outer spring end 20h is formed in one piece from the end portion of the spring band. The outermost turn of the spring band is connected (for example by spot welding or stamping or by mechanical anchoring) to a sleeve-shaped ring, whereby a spring housing is formed integrally from the spring band.

The display element 25a is positioned axially to the latter by its support surface 25e, which is in contact with the correspondingly configured contact surface 20k, and by its holding cams 25f, which are in engagement with the correspondingly configured holding cams 201 of the spring coil 20b. The rotational positioning of the display element 25a is achieved by the anti-twisting ribs 20m of the spring coil 20b, which are in engagement with the corresponding anti-twisting grooves 25g of the display element 25a.

The display window 25b is axially fixedly positioned relative to the mechanism holder 13 by its holding openings 25h, which engage with the correspondingly configured holding cams 13v, and by its support surface 25i, which is in contact with the correspondingly configured contact surface 13w of the mechanism holder 13. The rotational positioning of the display window 25b is achieved by its anti-twisting grooves 25j, which engage with the correspondingly configured anti-twisting ribs 13x of the mechanism holder 13.

The display cap 25c is axially non-detachably and rotationally fixedly positioned by its holding catches 25k, which are in engagement with the correspondingly configured holding openings 25l, as well as by its support surface 25m, which is in contact with the correspondingly configured contact surface 25n.

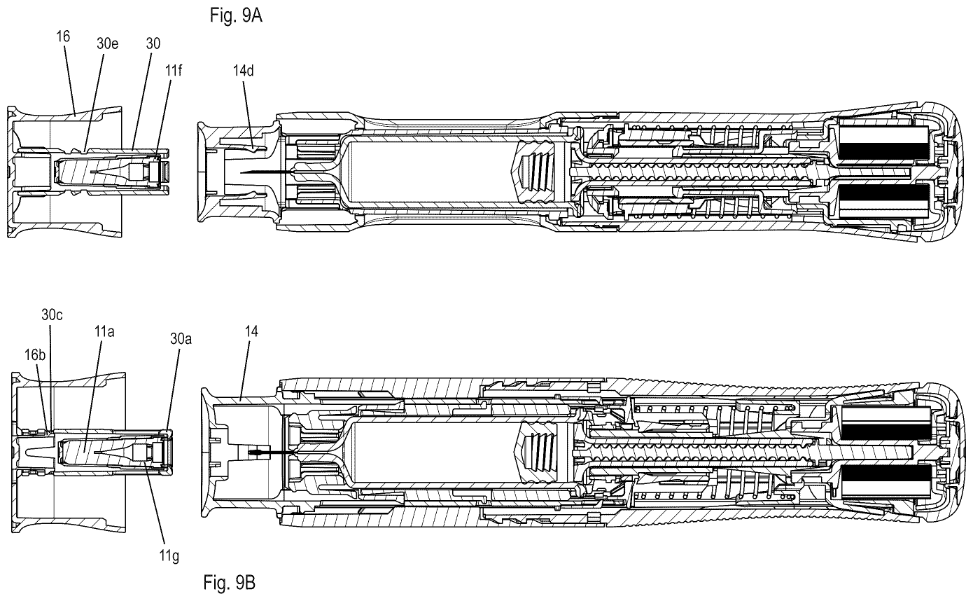

FIGS. 8A and 8B show two longitudinal cross-sections of the autoinjector from FIGS. 5A and 5B rotated by 90° relative to one another in the state of the trigger lock, which can be activated, for example, by a strong acceleration of the autoinjector.

When the needle protection sleeve 14 is moved in the proximal direction, the holding catches 14d are deflected via the holding openings 30e on the insert 30. The locking surface 14j abuts the locking surface 12j of the syringe holder 12, which is delimited in the proximal direction by contact of the holding catches 12f on the support surfaces 10k of the syringe housing part 10b. By means of the locking catches 17e, which engage in the holding openings 14k, the proximal movement of the switching sleeve 17 and thus also of the locking sleeve 18 is delimited to such an extent that the holding arms 23e of the coupling sleeve 23 are always locked against deflection. This ensures that the injection cannot be triggered as long as the device cap 16 has not been removed.

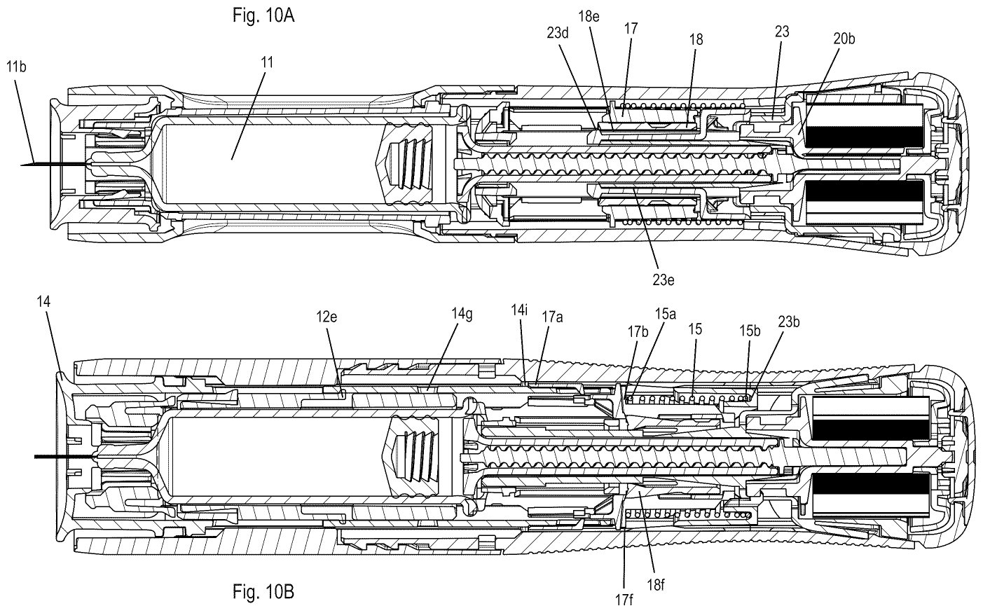

FIGS. 9A and 9B show two longitudinal sections of the autoinjector from FIGS. 5A and 5B, rotated by 90° relative to one another, in the removed state of the device cap including the needle protection cap.

When in use, the device cap 16 is pulled off the autoinjector in a distal direction. The insert 30, which is permanently connected to the device cap 16 by its holding openings 30c and the holding cam 16b of the device cap, is removed together with the device cap 16. In this case, the connection between the holding catches 14d of the needle protection sleeve 14 and the correspondingly configured holding openings 30e of the insert 30, which can be released with the application of force, is released.

The needle protection cap 11a, which is permanently connected to the insert 30 by its shoulder 11f and the holding arms with holding cams 30a of the insert, is removed together with the device cap 16 and the insert 30. In this case, the clamping region 11g between the needle protection cap 11a and the ready-to-use syringe 11, which can be released with the application of force, is released.

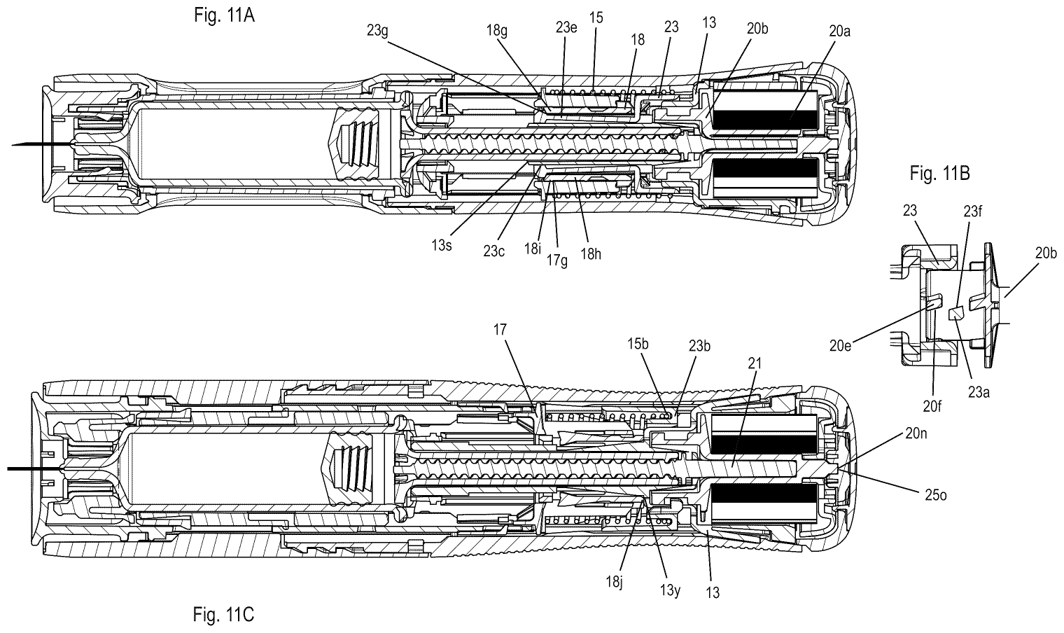

FIGS. 10A and 10B show two longitudinal cross-sections of the autoinjector from FIGS. 5A and 5B rotated by 90° relative to one another in the inserted state, wherein the torque on the spring coil 20b is still blocked.

In order to insert the injection needle 11b of the ready-to-use syringe 11 into the injection site, the autoinjector is pressed with its distal end onto the injection site during use. During the needle insertion process, the needle protection sleeve 14 is displaced in the proximal direction, whereby the needle previously covered by the needle protection sleeve 14 is inserted into the skin. At the beginning of the needle insertion process, a defined trigger resistance must be overcome. For this purpose, the connection between the holding cams 12e of the syringe holder 12 and the corresponding holding openings 14g of the needle protection sleeve 14, releasable using force, is released. The switching sleeve 17, which, with its support surfaces 17a, is in contact with the corresponding contact surfaces 14i of the needle protection sleeve 14, is displaced together with the needle protection sleeve 14 in the proximal direction. In this case, the needle protection spring 15, which rests with its distal spring end 15a on the correspondingly configured spring support 17b of the switching sleeve 17 and with its proximal spring end 15b on the correspondingly configured spring support 23b of the coupling sleeve 23, is preloaded during the displacement of the needle protection sleeve 14 and the switching sleeve 17. The locking sleeve 18, which is in contact with the correspondingly configured holding surfaces 17f of the switching sleeve 17 with its locking cams 18f, is displaced in the proximal direction together with the needle protection sleeve 14 and the switching sleeve 17. By displacing the locking sleeve 18 relative to the coupling sleeve 23, the contact surfaces 18e of the locking sleeve 18 are moved away from the contact surfaces 23d of the holding arms 23e of the coupling sleeve 23, whereby the holding arms 23e of the coupling sleeve 23 are no longer secured against deflection and can therefore deflect outwards.

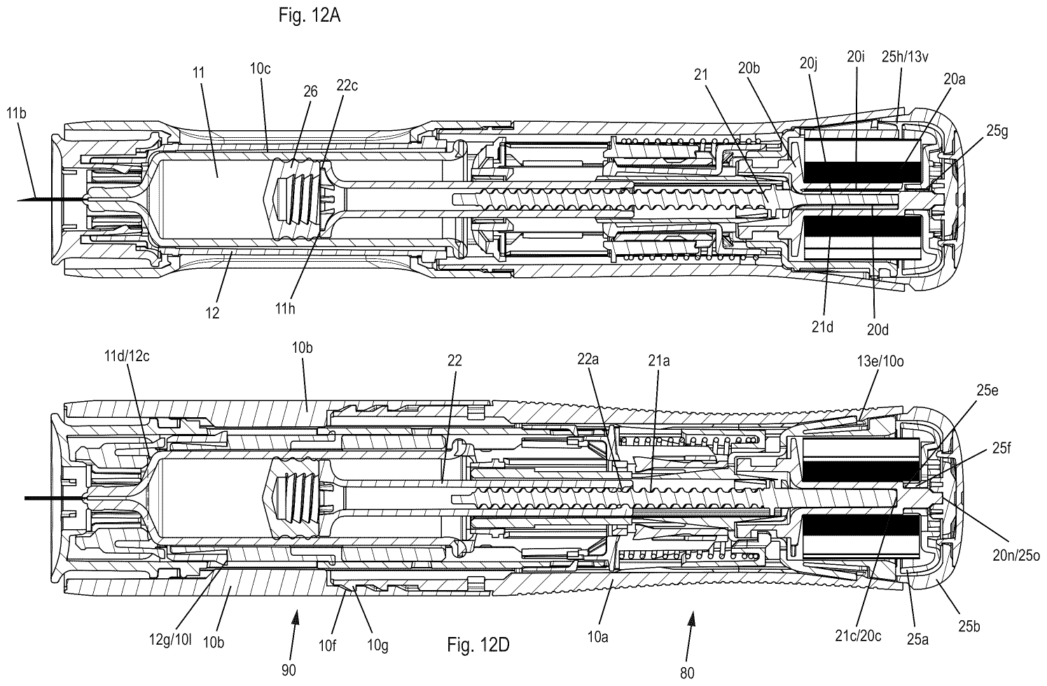

FIGS. 11A and 11C shows two longitudinal sections of the autoinjector from FIGS. 5A and 5B rotated by 90° relative to one another in the drive release state, and FIG. 11B shows a partial sectional view of the coupling sleeve and the spring coil of the autoinjector from FIGS. 5A and 5B in the drive release state, immediately after the spring energy is released.

Due to the spring force of the needle protection spring 15, which has contact with its proximal spring end 15b to the correspondingly configured spring support 23b of the coupling sleeve 23, the coupling sleeve 23 is displaced in the proximal direction relative to the mechanism holder 13 and the spring coil 20b. In this case, the holding arms 23e of the coupling sleeve 23, which were previously released by the locking sleeve 18, deflect outwards through their inwardly directed, inclined holding cams 23c, which previously had contact with the correspondingly configured holding surfaces 13s of the mechanism holder 13, whereby the outwardly directed holding surfaces 23g now come into contact with the correspondingly configured holding surfaces 18g of the holding arms 18h of the locking sleeve 18. The contact between the contact surfaces 18i and the correspondingly configured contact surfaces 17g of the switching sleeve 17 prevents the holding arms 18h of the locking sleeve 18 from spreading outwards, whereby the coupling sleeve 23 is held in position relative to the mechanism holder 13 and the spring coil 20b by the locking sleeve 18. Due to the contact between the outwardly directed holding surfaces 23g of the coupling sleeve 23 and the correspondingly arranged holding surfaces 18g of the holding arms 18h of the locking sleeve 18, the latter is pulled by the coupling sleeve 23 in the proximal direction relative to the mechanism holder 13 until its support surfaces 18j rest against the correspondingly configured support surfaces 13y of the mechanism holder 13.

Due to the displacement of the coupling sleeve 23 relative to the spring coil 20b in the proximal direction, the coupling between the contact surfaces 23f of the coupling cams 23a of the coupling sleeve 23 and the correspondingly configured contact surfaces 20f of the distally arranged coupling cams 20e of the spring coil 20b has been released, whereby the stored spring energy of the spiral spring 20a is now released; FIG. 11B. Since the spring coil 20b is now no longer rotationally blocked by the coupling to the coupling sleeve 23 and is thus no longer axially preloaded relative to the mechanism holder 13, the spring coil 20b is displaced relative to the mechanism holder 13 and the display window 25b in the proximal direction by the counterforce of the drive element 21 until its support surface 20n rests against the correspondingly configured support surface 250 of the display window 25b.

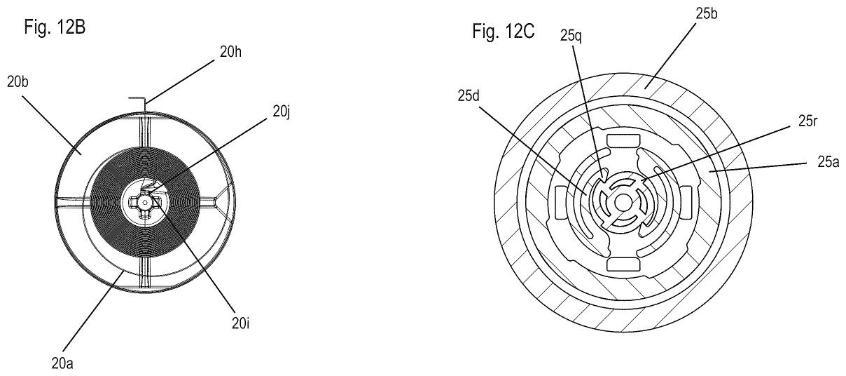

FIGS. 12A and 12D show two longitudinal cross-sections of the autoinjector from FIGS. 5A and 5B in the delivery state, rotated by 90° relative to one another, FIG. 12B shows a view proximal to the spring coil 20b with the spiral spring (also called mainspring) 20a of the autoinjector 1 from FIGS. 5A and 5B, and FIG. 12C shows a cross section from a distal view of the display element 25a with display window 25b of the autoinjector 1 from FIGS. 5A and 5B. The spring energy stored in the spiral spring 20a is passed on to the spring coil 20b via its inner spring end 20i, which is in engagement with the correspondingly configured spring receptacle 20j, whereby the spring coil 20b rotates. The spring coil 20b transmits its rotational movement to the drive element 21 via its anti-twisting surfaces 20d, which are in contact with the correspondingly configured anti-twisting surfaces 21d of the drive element 21; FIGS. 12A and 12B. The drive element 21 in turn drives the propulsion element 22 in the distal direction via its external threaded portion 21a, which is in contact with the correspondingly configured internal threaded part 22a of the propulsion element 22, wherein rotation of the propulsion element 22 is prevented by its outer anti-twisting ribs 22b, which are in engagement with the correspondingly configured internal anti-twisting grooves 13h (not shown) of the mechanism holder.

The stopper 26 in the ready-to-use syringe 11, which has contact with its contact surface 11h to the correspondingly configured support surface 22c of the propulsion element 22, is displaced together with the propulsion element 22 in the distal direction, whereby the medication provided in the ready-to-use syringe 11 is delivered through its injection needle 11b.

During the ongoing injection, the injection progress is visible to a user through the viewing window 10c of the syringe housing part 10b by looking at the stopper 26. Since the injection speed can be very slow and therefore difficult to perceive visually (e.g., 60 seconds injection time for a fully filled ready-to-use syringe), an additional injection movement indicator is integrated at the proximal end of the autoinjector. The display element 25a, with its support surface 25e abutting the correspondingly configured contact surfaces 20k of the spring coil, axially snapped to the holding cams 201 of the spring coil 20b by means of holding cams 25f and engaging through its anti-twisting grooves 25g in the anti-twisting ribs 20m of the spring coil 20b, rotates together with the spring coil 20b relative to the display window 25b during the injection. This rotational movement of the display element 25a is visible to a user through the transparent display window 25b by an applied pattern 25p, thereby providing a user with additional and improved visual feedback on the ongoing injection.

In addition to the visual feedback, a user receives continuous acoustic feedback on the ongoing injection in the form of clicking sounds generated between the rotating display element 25a and the stationary display window 25b. The snap-in cams 25q attached to the radially deflectable spring arms 25d of the display element 25a slide during the injection over the correspondingly radially configured snap-in grids 25r of the display window 25b, wherein the spring arms 25d of the display element 25a are radially pre-tensioned and relaxed again during the rotation relative to the display window 25b, whereby a continuous, acoustic clicking sound becomes perceptible for a user. If the click frequency is to be adapted to a delivery speed, the number of grids 25r can be varied. In this case, it is important to ensure that the pitch corresponds to the assembly positions of the spring arms 25d. For example, 2, 4 or 8 grids per revolution would be conceivable.—FIG. 12C shows an embodiment with 4 grids 25r and two spring arms 25d.

The injection force pushes the two subassemblies, product container receiving device 90 and drive device 80, apart, which are held together via the double snap groove 10f and double holding cam 10g on the syringe housing part 10b and drive housing part 10a, respectively. In the distal direction, the injection force is transmitted via the interfaces 11d/12c and 12g/10l from the ready-to-use syringe 11 via the syringe holder 12 to the syringe housing part 10b. In the proximal direction, the injection force is transmitted via the interfaces 22a/21a, 21c/20c, 20n/25o, 25h/13v and 13e/10o from the propulsion element 22 via the drive element 21, the spring coil 20b, the display window 25b and the mechanism holder 13 to the drive housing part 10a.

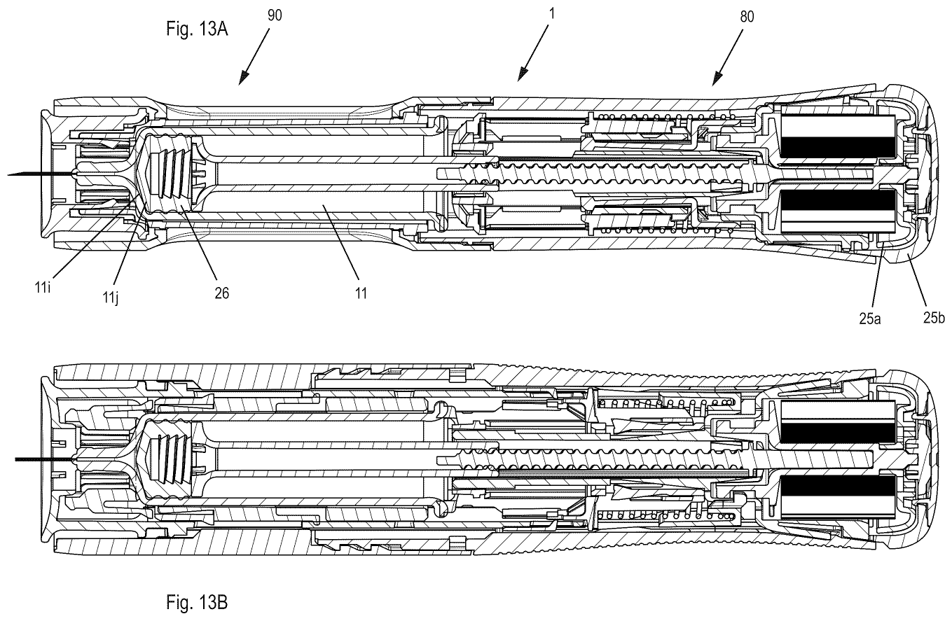

FIGS. 13A and 13B show two longitudinal cross-sections of the autoinjector from FIGS. 5A and 5B, rotated by 90° relative to one another, in the delivery completed state.

The displacement of the stopper 26 in the distal direction stops automatically as soon as the stopper 26, with its contact surface 11j arranged at the distal end, strikes the correspondingly configured contact surface 11i of the ready-to-use syringe 11.

The automatic injection is thus finished. The completed delivery is visible to a user through the motionless display element 25a and the absence of the clicking sound, which is caused by the relative movement of the display element 25a and the display window 25b that is fixed to the housing.

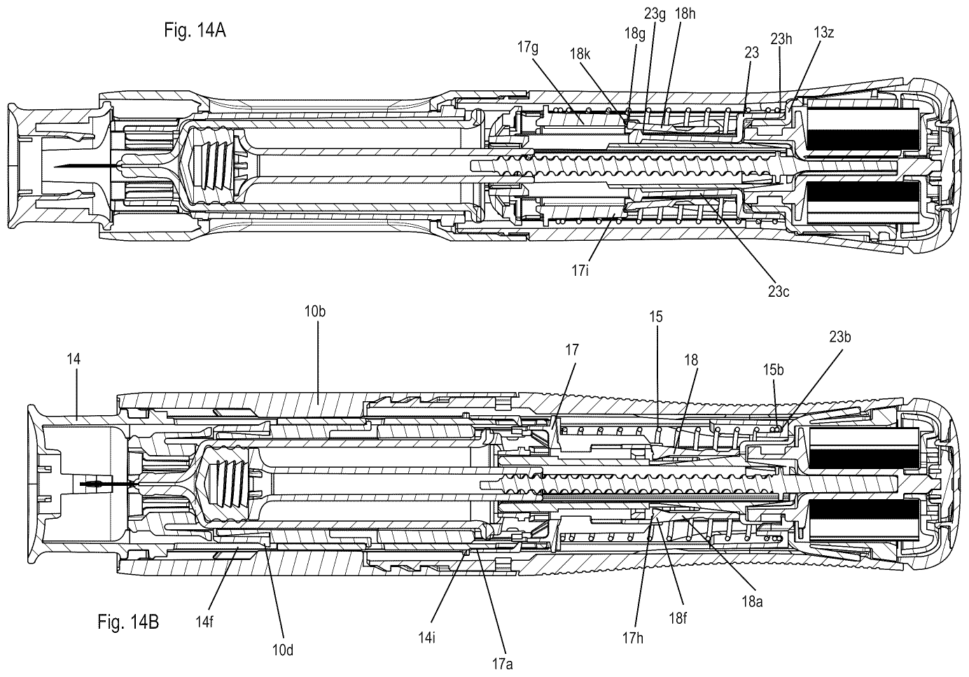

FIGS. 14A and 14B show two longitudinal cross-sections of the autoinjector from FIGS. 5A and 5B, rotated by 90° relative to one another, in the activated state of the needle protection lock.

After the injection has taken place or the delivery has ended, the needle protection sleeve 14 is displaced back into its original position in the distal direction by the spring force of the needle protection spring 15, together with the switching sleeve 17, and is blocked by the locking sleeve 18 against further insertion. The outwardly directed locking cams 18f of the locking arms 18a, as well as the locking surfaces 18k of the outwardly deflected holding arms 18h of the locking sleeve 18, come into contact with the correspondingly configured locking surfaces 17h and the locking surfaces 17i of the switching sleeve 17, whereby a further displacement of the switching sleeve 17 and thus of the needle protection sleeve 14 in the proximal direction is prevented by the support surfaces 17a being in positive contact with the correspondingly configured contact surfaces 14i.

During removal of the autoinjector 1 from the injection site, the needle protection sleeve 14 and the switching sleeve 17 are displaced distally back into their original positions by the spring force of the needle protection spring 15, which is in contact with the correspondingly configured spring support 17b of the switching sleeve 17 with its distal spring end 15a and which switching sleeve is in turn is in contact with the correspondingly configured contact surfaces 14i of the needle protection sleeve 14 through its support surfaces 17a, until the holding openings 14f of the needle protection sleeve 14 come back into contact with the holding cams 10d of the syringe housing part 10b.

The switching sleeve 17 is displaced by the spring force of the needle protection spring 15 together with the needle protection sleeve 14 in the distal direction to its original position. During the displacement of the switching sleeve 17 relative to the locking sleeve 18, the locking arms 18a of the locking sleeve 18 are briefly deflected inwards until the switching sleeve 17 has completely moved away from the locking cams 18f of the locking arms 18a, whereby the locking cams 18f of the locking arms 18a now come to lie behind the proximal end of the switching sleeve 17.

The displacement of the switching sleeve 17 relative to the locking sleeve 18 causes its holding arms 18h, which, via the contact surfaces 18i thereof, previously had contact with the correspondingly arranged contact surfaces 17g of the switching sleeve 17 and were thus secured against deflection, to now be able to deflect outwards. The coupling sleeve 23, which is secured against displacement by the holding surfaces 23g of the holding arms 23e of the coupling sleeve 23, which previously had contact with the correspondingly configured holding surfaces 18g of the holding arms 18h of the locking sleeve 18 thus secured against displacement, can now be displaced in the proximal direction relative to the locking sleeve 18 and the mechanism holder 13.

Due to the spring force of the needle protection spring 15, which, with its proximal spring end 15b, is in contact with the correspondingly configured spring support 23b of the coupling sleeve 23, the coupling sleeve 23 is now displaced in the proximal direction relative to the locking sleeve 18 until its support surfaces 23h rest against the correspondingly configured support surfaces 13z of the mechanism holder 13. In this case, the holding arms 18h of the locking sleeve 18 are deflected outwards by the holding surfaces 23g of the holding arms 23e of the coupling sleeve 23.

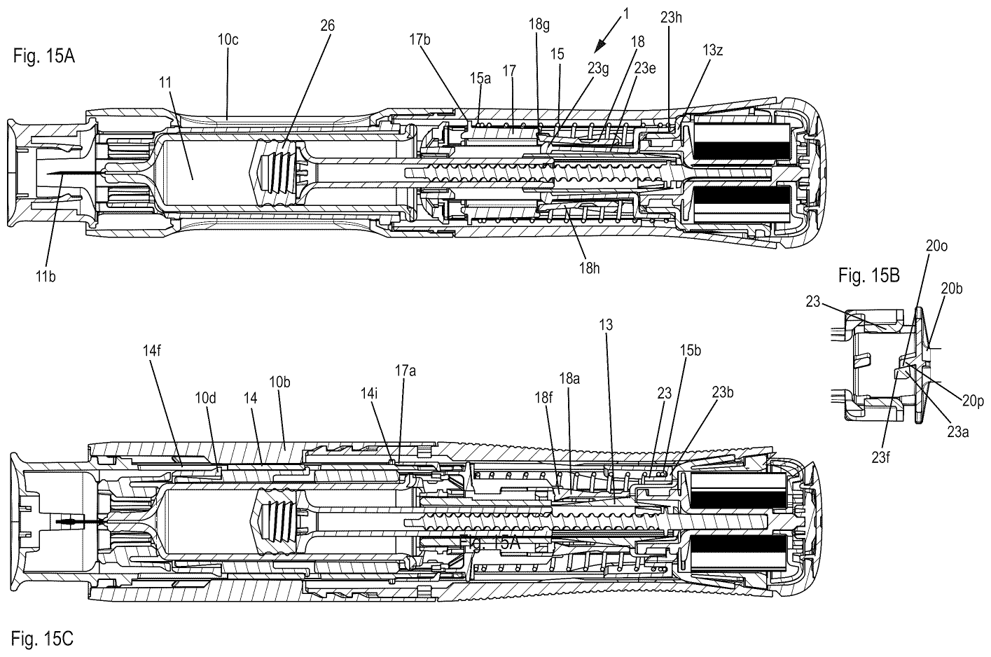

FIGS. 15A and 15C show two longitudinal cross-sections of the autoinjector from FIGS. 5A and 5B rotated by 90° relative to one another in the delivery state, and FIG. 15B shows a partial sectional view of the coupling sleeve and the spring coil of the autoinjector from FIGS. 5A and 5B in the delivery state.

An injection abort or a stop of the delivery occurs if a user removes the autoinjector 1 from the injection site before the delivery is complete, which also activates the automatic needle protection lock. At the same time, the automatic delivery is stopped, thus preventing unwanted dripping of the remaining medication from the injection needle 11b.

During removal of the autoinjector from the injection site, the needle protection sleeve 14 and the switching sleeve 17 are displaced distally back into their original positions by the spring force of the needle protection spring 15, which is in contact with the correspondingly configured spring support 17b of the switching sleeve 17 with its distal spring end 15a and which switching sleeve in turn is in contact with the correspondingly configured contact surfaces 14i of the needle protection sleeve 14 through its support surfaces 17a, until the holding openings 14f of the needle protection sleeve 14 come back into contact with the holding cams 10d of the syringe housing part 10b.

The switching sleeve 17 is displaced by the spring force of the needle protection spring 15 together with the needle protection sleeve 14 in the distal direction to its original position. During the displacement of the switching sleeve 17 relative to the locking sleeve 18, the locking arms 18a of the locking sleeve 18 are briefly deflected inwards until the switching sleeve 17 has completely moved away from the locking cams 18f of the locking arms 18a, whereby the locking cams 18f of the locking arms 18a now come to lie behind the proximal end of the switching sleeve 17.

The displacement of the switching sleeve 17 relative to the locking sleeve 18 causes its holding arms 18h, which, via the contact surfaces 18i thereof, previously had contact with the correspondingly arranged contact surfaces 17g of the switching sleeve 17 and were thus secured against deflection, to now be able to deflect outwards. The coupling sleeve 23, which is secured against displacement by the holding surfaces 23g of the holding arms 23e of the coupling sleeve 23, which previously had contact with the correspondingly configured holding surfaces 18g of the holding arms 18h of the locking sleeve 18, can now be displaced in the proximal direction relative to the locking sleeve 18 and the mechanism holder 13.

Due to the spring force of the needle protection spring 15, which, with its proximal spring end 15b, is in contact with the correspondingly configured spring support 23b of the coupling sleeve 23, the coupling sleeve 23 is now displaced in the proximal direction relative to the locking sleeve 18 until its support surfaces 23h rest against the correspondingly configured support surfaces 13z of the mechanism holder 13. In this case, the holding arms 18h of the locking sleeve 18 are deflected outwards by the holding surfaces 23g of the holding arms 23e of the coupling sleeve 23.

The displacement of the coupling sleeve 23 in the proximal direction relative to the spring coil 20b causes its contact surfaces 23f of the coupling cams 23a to come into engagement with the correspondingly configured contact surfaces 200 of the proximally arranged coupling cams 20p of the spring coil 20b; FIG. 15B. This blocks the rotation of the spring coil 20b and stops the automatic delivery.

After an injection has been aborted by a user, the axial position of the stopper 26 of the ready-to-use syringe 11 can be seen through the viewing window 10c of the syringe housing part, whereby a user can estimate how much injection volume has been delivered or how much residual medication is still left.

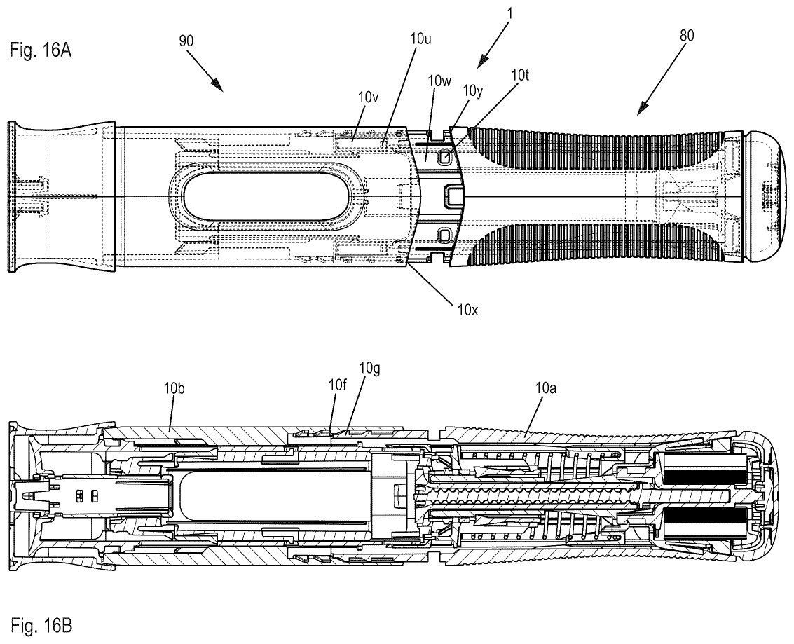

FIGS. 16A and 16B show a view and a longitudinal cross-section of the autoinjector 1 from FIGS. 5A and 5B in the pre-snapped state. The pre-snapped state is the initial state for the assembly of a ready-to-use syringe and the final assembly of the autoinjector 1. In the pre-snapped state, the two subassemblies, drive device 80 and product container receiving device 90, are separably connected to one another and can be safely stored and transported in a defined position relative to one another due to the force-fitting axial and form-fitting rotational fixation. The subassemblies product container receiving device 90 and drive device 80 are pre-snapped by means of the detachable snap connection 10v/10u between syringe housing part 10b and drive housing part 10a. For this purpose, the snap-in cam 10u on the drive housing part 10a engages behind the proximal edge of the snap-in groove 10v in the syringe housing part 10b. Furthermore, the distal side of the double holding cam 10g abuts the proximal edge of the double holding groove 10f and thus positions the housing parts 10a, 10b axially relative to one another at a defined distance as shown in FIGS. 16A and 16B. A peripheral step 10y introduces a distal end portion 10w on the drive housing part 10a. The assembly windows 10t in the distal end portion 10w of the drive housing part 10a are visible and accessible from the outside. The end portion 10w is partially inserted into the proximal end of the syringe housing part 10b, wherein a peripheral axially acting gap guide 10x is formed between the two housing parts 10a, 10b in the region of the end portion 10w.

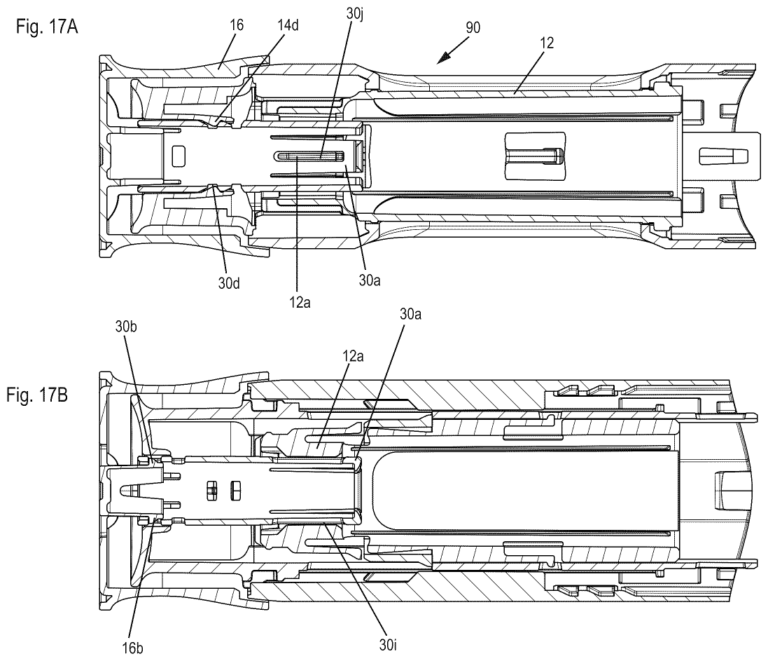

FIGS. 17A and 17B show two longitudinal cross-sections of the product container receiving device 90 with insert 30 pre-assembled, rotated by 90° relative to one another. In a first step, the drive device 80 was removed from the pre-snapped autoinjector and the product container receiving device 90 is thus ready to receive a ready-to-use syringe in one joining process through the proximal opening. In the pre-assembled state, the insert 30 is in engagement with its distal holding openings 30b with the correspondingly configured holding cams 16b of the device cap 16. In addition, the holding catches 14d of the needle protection sleeve 14, which can be released with the application of force, engage in the correspondingly configured holding openings 30d of the insert 30, whereby the latter is positioned in relation to the other parts of the product container receiving device 90. In this state, the holding slots 30j of the holding arms 30a are positioned in relation to the correspondingly arranged holding ribs 12a of the syringe holder 12 in such a way that the flexible holding arms with the holding cams 30a can deflect outwards, wherein the holding ribs 12a of the syringe holder 12 move into the holding slots 30j of the holding arms 30a or the holding arms 30a find space in the axially running recesses next to the holding ribs 12a.

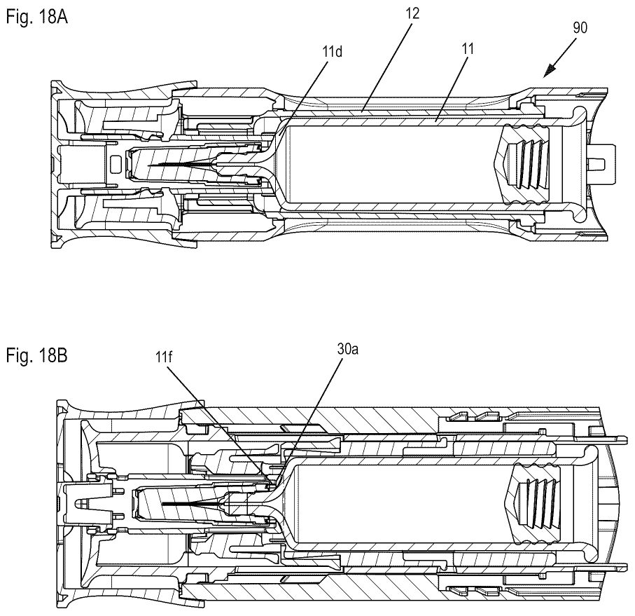

FIGS. 18A and 18B show two longitudinal cross-sections of the product container receiving device 90 with pre-assembled ready-to-use syringe 11 (PFS with RNS), rotated by 90° relative to one another. The ready-to-use syringe 11 is inserted axially in the distal direction into the product container receiving device 90 until its support surface 11d comes into contact with the correspondingly configured holding arms with holding cams 30a and the support surfaces 30h of the insert 30. During this joining process of the ready-to-use syringe 11, the holding cams of the flexible holding arms 30a of the insert 30 slide over the outer surface of the needle protection cap 11a of the ready-to-use syringe 11 until the holding cams on the holding arm 30a resiliently snap into place or come to rest proximal to the shoulder 11f of the needle protection cap 11a.

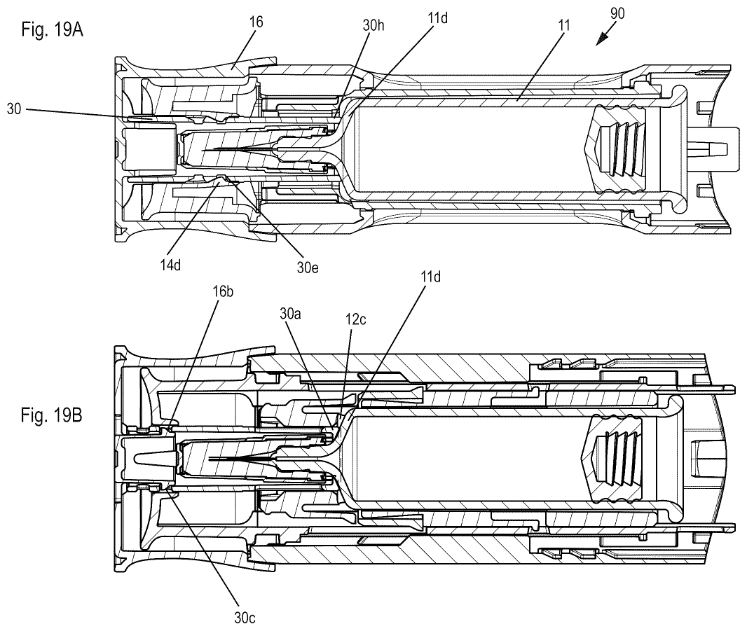

FIGS. 19A and 19B show two longitudinal sections of the product container receiving device 90 with insert 30 and ready-to-use syringe 11 (PFS with RNS) rotated by 90° relative to one another in the fully assembled state. For this final assembly step, the ready-to-use syringe 11 is inserted further axially in the distal direction into the autoinjector until its support surfaces or shoulders 11d come into contact with the correspondingly arranged support edges 12c of the syringe holder 12. Due to the contact of the support surface 11d of the ready-to-use syringe 11 with the corresponding holding cams 30a and the front support surfaces 30h of the insert 30, the latter is pushed in the distal direction relative to the device cap 16. In this case, the holding cams 16b change from the distal holding openings 30b to the proximal holding openings 30c. In addition, the holding catch 14d of the needle protection sleeve 14 changes from the holding opening 30d into the correspondingly configured proximal holding openings 30e of the insert 30. As a result of this final assembly step, the holding arms with the holding cams 30a now pass under the holding ribs 12a of the syringe holder 12, or the holding slots 30j of the holding arms no longer expose the holding ribs 12a of the syringe holder 12, which prevents the flexible holding arms with the holding cams 30a of the insert 30 from deflecting outwards. Thus, the engagement of the holding cams 30a with the RNS shoulder 11f of the ready-to-use syringe 11 is secured in a form-fitting manner.

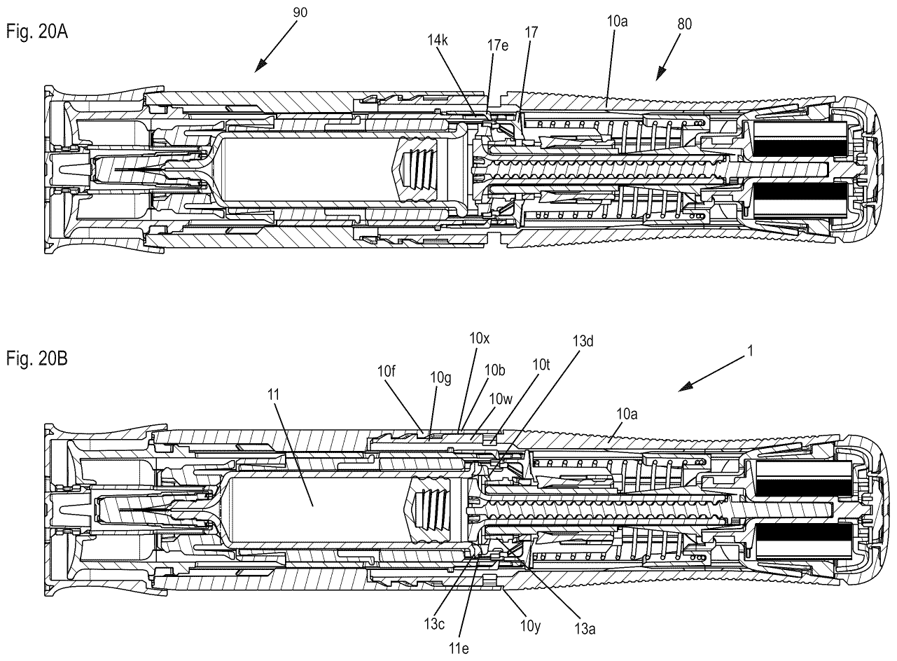

FIG. 20A shows a longitudinal cross-section of the product container receiving device 90 with assembled ready-to-use syringe 11 after a first joining step of the drive device 80. During this first joining step, an assembly tool (not shown) can be inserted laterally through the assembly window 10t (see FIGS. 16A and 16B) and fix the switching sleeve 17 axially relative to the housing part 10a by engaging in the assembly groove 17j (see FIG. 6), thus ensuring that the locking catch 17e on the firmly held switching sleeve 17 can deflect into the holding opening 14k of the needle protection sleeve 14 during the first joining step.

FIG. 20B shows a longitudinal cross-section of the product container receiving device 90 with assembled ready-to-use syringe 11 after a second final joining step of the drive device 80. Before this second joining step, the assembly tool (not shown) is removed.

In this second joining step, the drive device 80 is pushed axially in a linear movement along the gap guide 10x into the product container receiving device 90 until it stops at the step 10y, wherein the double holding cams 10g snap into their respective corresponding double snap grooves 10f and connect the drive device 80 in a form-fitting and thus non-detachable manner to the product container receiving device 90. As a result of the second joining step, the assembly windows 10t at the distal end portion 10w of the drive housing part 10a are covered by the peripheral sleeve-shaped end of the syringe housing part 10b, whereby the outer shell of the fully assembled auto-injector 1 is not broken through by disturbing openings and the interior of the autoinjector 1 remains protected. The support surface 13c of the syringe adapter 13a is axially positioned by the support surface Ile at the proximal end of the ready-to-use syringe 11, wherein the flexible elements 13d of the syringe adapter 13a, to which the support surface 13c is connected, are pre-loaded by the second joining step.

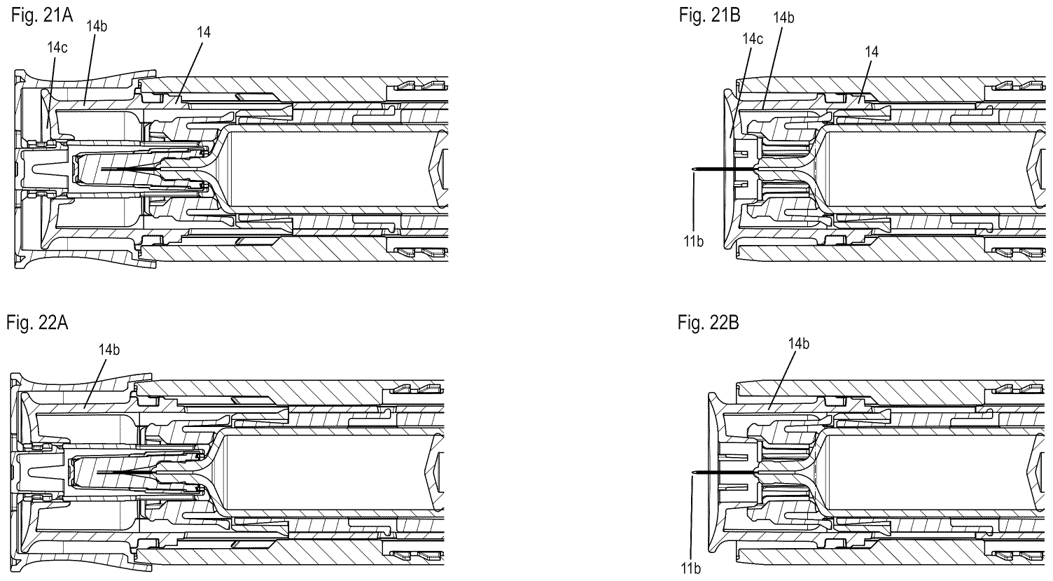

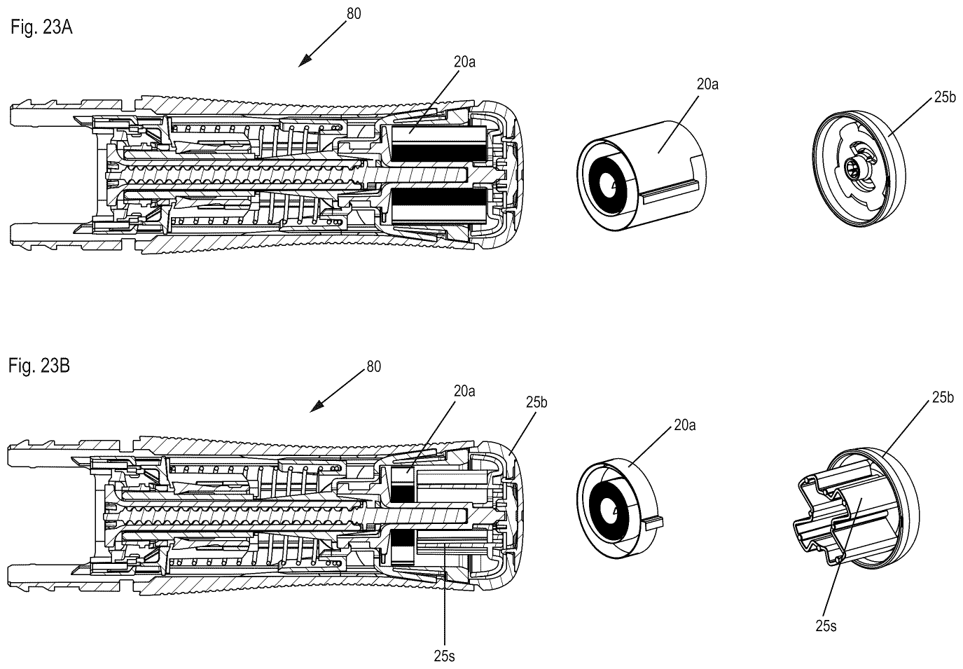

With autoinjector variants, it should be possible to insert the needle into the skin to a depth of, for example, about 3-15 mm. FIG. 21A shows an autoinjector variant for a piercing depth by way of example of 8 mm in the delivery state, and FIG. 21B in the inserted state. FIG. 22B shows an autoinjector variant for a piercing depth by way of example of 5 mm in the delivery state, and FIG. 22B in the inserted state. These two and other variants differ only in terms of different needle protection sleeves 14, in each of which the axially extending portion 14b is shorter or longer, whereby the flange 14c to be placed on the skin is positioned differently, whereby the piercing depth of the needle 11b is delimited accordingly.

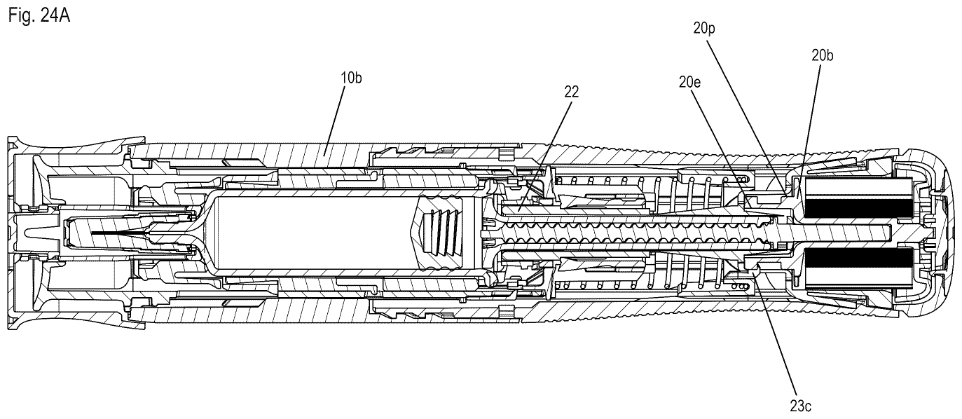

FIGS. 23A and 23B each show a longitudinal cross-section through the proximal end of the drive device 80 as well as a view of the mainspring 20a and the display window 25b. In order to achieve different torques or injection forces, different mainsprings can be installed. In this case, the mainsprings have different spring band widths and therefore deliver different spring torques. Complementary to the spring band width, axially extending filling profiles 25s of different lengths can be provided distally on the display window 25b so that the installation space for the spring is axially delimited. This prevents the spring coil from breaking out in the axial direction.

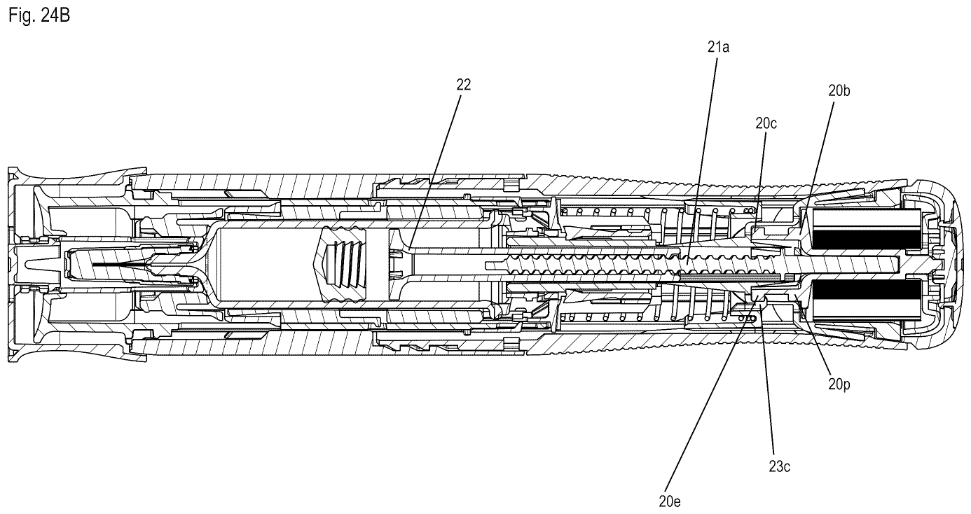

FIG. 24A and FIG. 24B each show a longitudinal section through autoinjectors with ready-to-use syringes, which are pre-filled with different volumes of medication. When the syringe is fully filled-FIG. 24A, the stroke of the propulsion element 22 is maximum. When the syringe is only partially filled-FIG. 24B, the stroke of the can be reduced by using different assembly positions, wherein the same parts can be used. The propulsion element 22 can be screwed onto the drive element 21 in a desired starting position. Gradations of the different starting positions for the propulsion element 22 result from its axially extending anti-twisting rib 22b, which is guided in the anti-twisting groove 13h of the mechanism holder 13 arranged correspondingly thereto. The gradation of the different starting positions of the propulsion element 22 is further dependent on the design of the coupling 23 or the spring coil 20b with regard to the number of holding cams 23c, distal coupling cams 20e, proximal coupling cams 20p, as well as on the thread pitch and the number of threads on the external threaded portion 21a on the drive element 21. In addition, an adapted syringe housing part 10b with shorter viewing windows 10c can be used for smaller filling volumes or smaller strokes of the propulsion element 22.

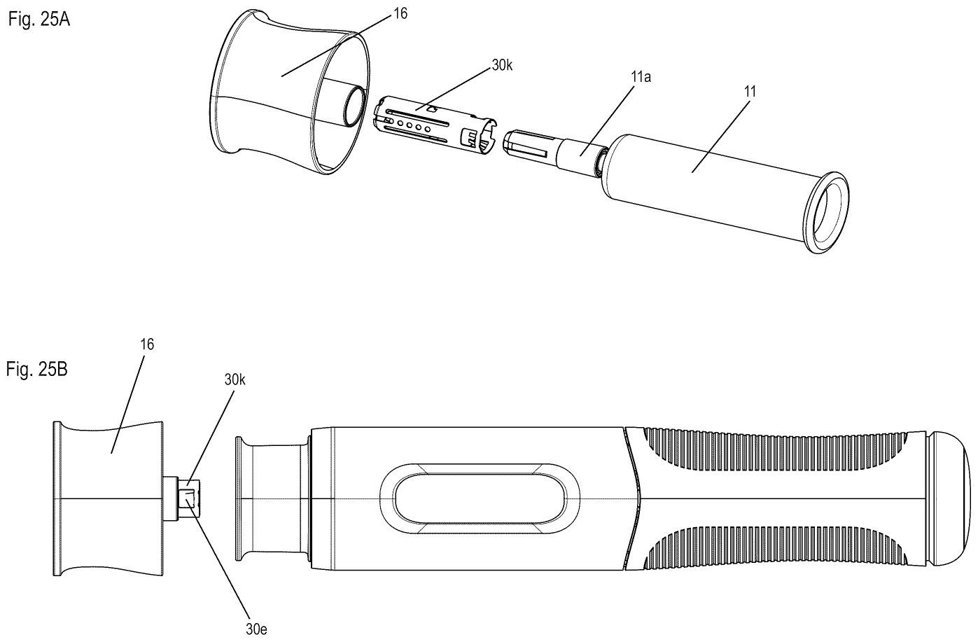

As shown in FIGS. 25A and 25B, as a variant, a release sleeve 30k with claws 301 inclined distally to the axis L can be fastened in a form-fitting manner, in particular irreversibly snapped, in the device cap 16 (instead of the insert 30). When joining the ready-to-use syringe, the claws 301 slide along the outer surface of the needle protection cap 11a. When the device cap 16 is removed, the claws grip the outer surface and take the needle protection cap 11a with them, thereby exposing the clamping region 11g and the injection needle 11b of the ready-to-use syringe 11.

LIST OF REFERENCE SIGNS

| | | Alt. | |
|---|---|---|---|
| Sign | Part | Abbreviation | Note |
| 1 | autoinjector | | |
| 10a | housing part, drive | DH | drive housing |
| | housing part | | |
| 10b | housing part, syringe | SHO | syringe housing |
| | housing part | | |
| 10c | recess, viewing window | SHO-h | |
| 10d | holding cam | SHO-a | |
| 10e | anti-twisting ribs | SHO-b | |
| 10f | double snap groove | SHO-c | |
| 10g | double holding cam | DH-a | |
| 10h | anti-twisting ribs | SHO-d | |
| 10i | anti-twisting surfaces | DH-b | |
| 10k | support surface | SHO-e | |
| 10l | support surface | SHO-f | |
| 10m | anti-twisting grooves | SHO-g | |
| 10n | viewing window edge | SHO-i | |
| 10o | holding cam | DH-d | |
| 10p | support surface | DH-e | |
| 10q | anti-twisting groove | DH-f | |
| 10r | guide rib | DH-c | |
| 10s | guide rib | DH-g | |
| 10t | assembly window | DH-i | |
| 10u | snap-in cam | DH-h | |
| 10v | snap-in groove | SHO-j | |
| 10w | end portion | | |
| 10x | gap guide | | |
| 10y | step | | |
| 11 | ready-to-use syringe | PFS | pre-filled syringe |
| 11a | needle protection cap | RNA | rigid needle shield |
| 11b | injection needle | | |
| 11c | cylindrical product container | | |
| 11d | shoulder, support surface | PFS-b | |
| 11e | support surface | PFS-c | |
| 11f | shoulder | PFS-a | on RNA |
| 11g | clamping region | PFS-d | |
| 11i | contact surface | PFS-e | |
| 11j | contact surface | PFS-f | |
| 11h | contact surface | PFS-g | on stopper 26 |
| 12 | syringe holder | SH | syringe holder |
| 12a | locking surface, holding ribs | SH-f | |
| 12b | guide sleeve | | |
| 12c | stop, support edge | SH-e | |
| 12d | recess | | only when rotating |
| 12e | holding cam | SH-a | |
| 12f | holding catch | SH-b | |
| 12g | support surfaces | SH-c | |
| 12h | anti-twisting ribs | SH-d | |
| 12i | anti-twisting ribs | SH-g | |
| 12j | locking surface | SH-h | |
| 13 | mechanism holder | MH | mechanics holder |
| 13a | holding spring portion; | SA | syringe adapter |
| | syringe adapter | | |
| 13b | spring sleeve | | |
| 13c | support surface | SA-a | |
| 13d | flexible elements | SA-b | |
| 13e | holding catch | MH-k | |
| 13f | support surface | MH-l | |
| 13g | anti-twisting ribs | MH-m | |
| 13h | anti-twisting grooves | MH-a | |
| 13i | holding catch | MH-b | |
| 13j | holding catch | SA-c | |
| 13k | holding opening | MH-c | |
| 13l | support surface | SA-d | |
| 13m | support surface | MH-d | |
| 13n | anti-twisting groove | SA-e | |
| 13o | anti-twisting rib | MH-e | |
| 13p | support surface | MH-f | |
| 13r | guide surface | MH-g | |
| 13s | holding surface | MH-h | |
| 13t | guide opening | MH-i | |
| 13q | support surface | MH-n | |
| 13u | spring receptacle | MH-o | |
| 13v | holding cam | MH-s | |
| 13w | contact surface | MH-t | |
| 13x | anti-twisting rib | MH-u | |
| 13y | support surface | MH-p | |
| 13z | support surface | MH-r | |
| 14 | needle protection sleeve | CS | cover sleeve |
| 14a | arm | | |
| 14b | portion | | |
| 14c | flange | | |
| 14d | holding catch | CS-b | |
| 14e | anti-twisting groove | CS-a | |
| 14f | holding openings | CS-c | |
| 14g | holding openings | CS-d | |
| 14h | anti-twisting grooves | CS-e | |
| 14i | contact surface | CS-f | |
| 14j | locking surface | CS-g | |
| 14k | holding opening | CS-h | |
| 15 | needle protection spring | CSS | cover sleeve |
| | | | spring |
| 15a | distal spring end | CSS-a | |
| 15b | proximal spring end | CSS-b | |
| 16 | device cap | CR | cap remover |
| 16a | rib, anti-twisting rib | CR-b | |
| 16b | snap cam, holding cam | CR-a | |
| 16c | snap-in cam | | only when rotating |
| 17 | switching sleeve | TOS | telescopic outer |
| | | | sleeve |
| 17a | support surface | TOS-a | |
| 17b | spring support | TOS-b | |
| 17c | guide surface | TOS-c | |
| 17d | holding opening | TOS-d | |
| 17e | locking catch | TOS-j | |
| 17f | holding surface | TOS-i | |
| 17g | contact surface | TOS-h | |
| 17h | locking surface | TOS-e | |
| 17i | locking surface | TOS-f | |
| 17j | assembly grooves | TOS-k | |
| 18 | locking sleeve | TIS | telescopic inner |
| | | | sleeve |
| 18a | locking member; | TIS-g | |
| | locking arms | | |
| 18b | holding cam | TIS-a | |
| 18c | holding cam | TIS-b | |
| 18d | guide surface | TIS-c | |
| 18e | contact surface | TIS-d | |
| 18f | locking cam | TIS-h | |
| 18g | holding surfaces | TIS-i | |
| 18h | holding arm | TIS-f | |
| 18i | contact surface | TIS-e | |
| 18j | support surface | TIS-k | |
| 18k | locking surface | TIS-l | |
| 20a | spiral spring, mainspring | DSP | drive spring |
| 20b | Spring coil | DS | |
| 20c | support surface | DS-a | |
| 20d | anti-twisting surface | DS-b | |
| 20e | coupling cam, distal | DS-e | |
| 20f | contact surface | DS-f | |
| 20g | support surface | DS-c | |
| 20h | outer spring end | DSP-b | |
| 20i | inner spring end | DSP-a | |
| 20j | spring receptacle | DS-d | |
| 20k | contact surface | DS-g | |
| 20l | holding cam | DS-h | |
| 20m | anti-twisting ribs | DS-i | |
| 20n | support surface | DS-k | |
| 20o | contact surface | DS-m | |
| 20p | coupling cam, proximal | DS-l | |
| 21 | drive element | TR | threaded rod |
| 21a | external threaded portion | TR-a | |
| 21b | peripheral shoulder | TR-b | |
| 21c | proximal support surface | TR-c | |
| 21d | anti-twisting surface | TR-d | |
| 22 | propulsion element | PR | plunger rod |
| 22a | internal threaded portion | PR-a | |
| 22b | anti-twisting ribs | PR-b | |
| 22c | support surface | PR-c | |
| 23 | coupling sleeve | CL | clutch |
| 23a | holding cams, coupling cams | CL-e | |
| 23b | spring support | CL-a | |
| 23c | holding cam | CL-b | |
| 23d | contact surface | CL-c | |
| 23e | holding arms | CL-d | |
| 23f | contact surface | CL-f | |
| 23g | holding surface | CL-g | |
| 23h | support surface | CL-h | |
| 23i | apron | | |
| 25a | display element | IR | indicator ring |
| 25b | window, display window | IW | indicator window |
| 25c | end, display cap | IC | indicator cap |
| 25d | engagement element, | IR-d | |
| | spring arm | | |
| 25e | support surface | IR-a | |
| 25f | holding cam | IR-b | |
| 25g | anti-twisting groove | IR-c | |
| 25h | holding opening | IW-a | |
| 25i | support surface | IW-f | |
| 25j | anti-twisting groove | IW-b | |
| 25k | holding catch | IC-a | |
| 25l | holding opening | IW-c | |
| 25m | support surface | IC-b | |
| 25n | contact surface | IW-g | |
| 25o | support surface | IW-d | |
| 25p | contrast pattern | IR-f | |
| 25q | snap-in cams | IR-e | |
| 25r | grid | IW-e | |
| 25s | filling profile | | |
| 26 | stopper | PL | plunger |
| 30 | insert | CRI | insert |
| 30a | snap arm with hook, holding | CRI-d | incl. CRI-g |
| | arm with holding cam | | |
| 30b | first snap groove, distal | CRI-e | |
| | holding opening | | |
| 30c | second snap groove, proximal | CRI-a | |
| | holding opening | | |
| 30d | first snap-fit, distal holding | CRI-h | |
| | opening | | |
| 30e | second snap-fit, proximal | CRI-c | |
| | holding opening | | |
| 30f | guide groove | | variant |
| 30g | groove, anti-twisting grooves | CRI-b | |
| 30h | end face, support surface | CRI-i | |
| 30i | Assembly cams | | variant |
| 30j | holding slots | CRI-f | |
| 30k | trigger sleeve | | variant |
| 30l | claws | | variant |
| 80 | drive device | DU | drive unit |
| 90 | product container receiving | SU | syringe unit |
| | device | | |
| L | longitudinal axis, axis | | |

What is claimed is:

1. An autoinjector, comprising:
a drive device having a first housing part of two housing parts, the drive device comprising a drive assembly;
a product container receiving device having a second housing part of the two housing parts, the product container receiving device defining a longitudinal axis and comprising a syringe holder configured to receive a pre-filled ready-to-use syringe with a cylindrical product container and a stopper movable along the longitudinal axis by the drive assembly for delivering a medication from the product container,
wherein the first housing part and the second housing part are configured to be connected coaxially in a joining direction in which a connection-side end portion on one of the two housing parts is stepped and configured to be inserted into another of the two housing parts, wherein a peripheral gap guide is formed between the two housing parts in a region of the end portion,
wherein in the region of the end portion, a first snap-in connection and a second and a third or final snap-in connection are provided, which can be snapped into place, one after the other, in the joining direction, and wherein the first snap-in connection is releasable against the joining direction and at least the third or final snap-in connection is locked.

2. The autoinjector according to claim 1, wherein the first housing part and the second housing part are configured to be connected by a linear movement parallel to the longitudinal axis in the joining direction or can be released counter to the joining direction.

3. The autoinjector according to claim 1, wherein a first, a second and a third or final snap-in position of the first housing part relative to the second housing part along the longitudinal axis is defined by the first snap-in connection and the second and the third or final snap-in connection.

4. The autoinjector according to claim 1, wherein the first, the second and the third or final snap-in connection are formed from cams and grooves formed on the first and the second housing parts in an interior of the gap guide.

5. The autoinjector according to claim 1, wherein one of the two housing parts with a radial step introducing the end portion is configured to abut peripherally against the connection-side end of the other of the two housing parts, wherein the radial step is configured to be continuously corrugated or discontinuously offset in a peripheral direction.

6. The autoinjector according to claim 5, wherein the one of the two housing parts with the radial step introducing the end portion abuts peripherally against the connection-side end of the other of the two housing parts when the third or final of the snap-in connections is locked.

7. The autoinjector according to claim 1, wherein the two housing parts comprise longitudinal guides in the region of the end portion and/or form a non-circular cross section of the gap guide.

8. The autoinjector according to claim 1, wherein one of the two housing parts comprises at least one assembly opening at an end portion configured to be accessible from an exterior at least in the first snap-in position and is configured to be completely covered by the other of the two housing parts in at least the third or final snap-in position.

9. The autoinjector according to claim 1, wherein the two housing parts can assume exactly three snap-in positions along the longitudinal axis.

10. The autoinjector according to claim 1, wherein the two housing parts are fastened to one another in the third or final snap-in position by the third or final snap-in connection and by gluing or welding.

11. The autoinjector according to claim 1, wherein the autoinjector further comprises a syringe adapter with a distal end configured to be abutted against a proximal end of the ready-to-use syringe, wherein the proximal end of the syringe adapter comprises a resilient element operatively connected to the first housing part.

12. The autoinjector according to claim 11, wherein the resilient element of the syringe adapter, to which a support surface is connected, is tensioned by the movement into the third or final snap-in position.

13. A method for a final assembly of an autoinjector, comprising the steps of:
providing an autoinjector comprising:
a drive device having a first housing part of two housing parts, the drive device comprising a drive assembly; and
a product container receiving device having a second housing part of the two housing parts, the product container receiving device defining a longitudinal axis and comprising a syringe holder configured to receive a pre-filled ready-to-use syringe,
wherein the first housing part and the second housing part are configured to be coaxially connected in a joining direction into a first of at least three snap-in positions, and are held in a first snap-in position for storage and/or transport purposes by a first snap-in connection configured to be released in and counter to the joining direction;
providing a ready-to-use syringe;
releasing the first snap-in connection of the two housing parts counter to the joining direction and separating the drive device from the product container receiving device;
inserting the ready-to-use syringe coaxially or axially parallel to the longitudinal axis into the product container receiving device;
joining the two housing parts in the joining direction into the first snap-in position defined by the first snap-in connection while leaving free an assembly opening on one of the two housing parts;
joining the two housing parts with the assembly opening exposed in the joining direction into a second of at least three snap-in positions which is held by a second snap-in connection of the two housing parts;
joining the two housing parts in the joining direction into a third or final snap-in position which is held and locked by a third or final snap-in connection of the two housing parts, and completely covering the assembly opening on one of the two housing parts by the other of the two housing parts when in the third or final snap-in position.

14. The method for the final assembly of an autoinjector according to claim 13, further comprising one or more of the following steps:
setting a starting position for a propulsion element by screwing the propulsion element onto a drive element;
holding and/or positioning a component movable inside one of the housing parts by means of an assembly tool that can be inserted through the exposed assembly opening;
removing the assembly tool from the assembly opening on one of the two housing parts before or when the two housing parts are in the second snap-in position, clamping a resilient syringe adapter when joining the two housing parts from the second to the third or final snap-in position.

15. The method for the final assembly of an auto-injector according to claim 13, wherein the two housing parts of the autoinjector are configured to be connected coaxially in a joining direction in which a connection-side end portion on one of the two housing parts is stepped and configured to be inserted into another of the two housing parts, wherein a peripheral gap guide is formed between the two housing parts in a region of the end portion,
wherein in the region of the end portion, the first snap-in connection and a second and a third or final snap-in connection are provided, which can be snapped into place, one after the other, in the joining direction, and wherein the first snap-in connection is releasable against the joining direction and at least the third or final snap-in connection is locked.
